(19) United States
(12) Patent Application Publication
(10) Pub. No.: US 2019/0374525 A1
(43) Pub. Date: Dec. 12, 2019

(54) TREATMENT OF DIABETES AND ASSOCIATED METABOLIC CONDITIONS WITH EPIGENETIC MODULATORS

(71) Applicant: Temple Otorongo LLC, Livingston, NJ (US)

(72) Inventors: Chris W. MAHNE, Livingston, NJ (US); Hasib SALAH-UDDIN, Miami, FL (US)

(21) Appl. No.: 16/485,330

(22) PCT Filed: Feb. 6, 2018
(86) PCT No.: PCT/US18/17074

§ 371 Date: Aug. 12, 2019

Related U.S. Application Data
(60) Provisional application No. 62/457,594, filed on Feb. 10, 2017.

(51) Int. Cl. A61K 31/4418; A61K 31/551; A61K 31/166; A61K 31/404; A61K 31/4725; A61K 31/5377; A61K 31/4406; A61K 31/473

(52) U.S. Cl. CPC A61K 31/4418; A61K 31/551; A61K 31/166; A61K 31/404; A61K 45/06; A61K 31/5377; A61K 31/4406; A61K 31/473; A61K 31/4725

(57) ABSTRACT

The present invention describes small molecules that have the activity of directly enhancing insulin sensitivity through epigenetic regulation. These small molecules, therefore, provide a new path for the treatment of type 2 diabetes and insulin resistance in type 1 diabetes or prediabetes and also can be used to treat obesity and chronic liver disease. Methods and compositions including these small molecules or for their administration are described. The methods and compositions can include additional anti-diabetic agents.

### CROSS-REFERENCE TO RELATED APPLICATION

This application claims the benefit of U.S. Provisional Application Ser. No. 62/457,594, by Chris W. Mahne and Hasib Salah-Uddin, filed on Feb. 10, 2017 and entitled “Treatment of Diabetes and Associated Metabolic Conditions with Epigenetic Modulators,” the contents of which are incorporated herein in their entirety by this reference.

### FIELD OF THE INVENTION

This invention is directed to treatment of diabetes and associated metabolic conditions, especially obesity and chronic liver disorders, using epigenetic modulators, including compositions and methods employing epigenetic modulators.

### BACKGROUND OF THE INVENTION

Type 2 diabetes is a metabolic disorder in which cellular uptake of glucose is impaired that causes blood glucose levels to rise higher than normal. In Type 2 diabetes, this is typically caused by the body not being able to utilize insulin properly; this is called insulin resistance. In prediabetes, a disorder that can lead to type 2 diabetes, insulin resistance is also a factor in which blood glucose levels rise higher than normal. Insulin resistance is also prevalent in type 1 diabetes, where autoimmune damage to beta-cells and thus reduced insulin secretion is change enough to manifest elevated blood glucose levels.

Long term complications for type 2 diabetes can include, but are not limited to, renal failure, peripheral neuropathy, diabetic retinopathy, cardiovascular complications, circulatory disorders, and reduced resistance to infections. Additionally, there is increasing evidence that type 2 diabetes can be associated with an increased frequency of Alzheimer's disease. Furthermore, evidence suggests that Alzheimer's disease represents a form of diabetes that selectively involves the brain and has molecular and biochemical features such as insulin resistance and insulin deficiency that overlap with both type 1 diabetes and type 2 diabetes and has been termed “type 3 diabetes.”

In the United States, diagnosis of type 2 diabetes has increased nearly sevenfold in the last 20 years, and the disease is being diagnosed at earlier and earlier ages. Patients who are diagnosed with type 2 diabetes at earlier than 60 years of age typically have poorer prognoses and more serious complications. Although part of this increase in the frequency of diagnosis may be due to increased awareness of the disease and more aggressive monitoring of patients' blood sugar levels who are deemed at risk for the disease, there is considerable evidence that the occurrence of type 2 diabetes has increased substantially. This may be due to obesity, diets high in refined sugars, high fructose corn syrup, and saturated fats, and a more sedentary lifestyle with decreased exercise patterns. There is also some evidence that genetic factors play a role, as the disease is more common in certain ethnic groups, such as African Americans, Native Americans, Hispanics, and other groups. There is definitely a genetic component, as having close blood relatives such as parents, uncles, or aunts with the disease definitely increases the risk.

Diabetes affects over 29 million Americans (10% of the population). The top 10 drugs currently on the market represent $28.6 billion in global annual sales. All of these drugs do not target the main underlying problem of enhancing insulin sensitivity in patients. The molecules identified using this strategy have the potential to fulfill an unmet need by treating the problem and not the symptom by enhancing insulin sensitivity.

A number of therapeutic approaches to the treatment of type 2 diabetes are currently in use. In some cases, insulin may be administered. However, this is neither indicated nor necessary in the vast majority of cases of type 2 diabetes, and the problem is generally not insufficient insulin, but the failure of the body to utilize the insulin that is present. A number of non-insulin therapeutic drugs are currently in use.

One commonly used class of therapeutic agents used to treat type 2 diabetes is the biguanides. The prototype of this class, and still one of the most commonly used agents for the treatment of diabetes, is metformin. The mechanism of action of metformin is not completely understood, but the drug appears to suppress hepatic gluconeogenesis and may increase insulin sensitivity. This agent is generally well tolerated, but may cause gastrointestinal symptoms and is not recommended in patients with significant liver or kidney problems. Other related biguanide drugs such as phenformin or buformin have been used, but have been withdrawn due to significant side effects.

Another class of therapeutic agents is the sulfonylureas. This class of agents includes acetohexamide, carbutamide, chlorpropamide, glycyclamide, metahexamide, tolazamide, tolbutamide, glibenclamide, glibomuride, gliclazide, glipizide, gliquidone, glisoxepide, glyclopyramide, and glimepiride. Although these agents may be effective in many cases of type 2 diabetes, they are associated with weight gain and may induce hypoglycemia, which can be severe. They are also subject to adverse interactions with many other classes of drugs.

Yet another class of antidiabetic agents is the thiazolidinediones, including pioglitazone and rosiglitazone. These agents are PPAR activators and act to decrease insulin resistance and to increase storage of fatty acids, forcing cells to utilize carbohydrates for oxidation. These agents have been linked to an increased risk of cardiovascular complications such as heart attack and stroke.

Still another class of antidiabetic agents is the DPP-4 inhibitors. These agents are inhibitors of dipeptidyl peptidase-4. These agents act to lower the levels of glucagon and reduce blood sugar levels. They also act to increase incretin levels, which act to promote insulin release. These agents include sitagliptin, vildagliptin, saxagliptin, linagliptin, gemigliptin, anagliptin, teneligliptin, alogliptin, trelagliptin, and omarigliptin. Although these agents are generally well-tolerated, they can produce a number of significant side effects, including nasopharyngitis, headache, nausea, heart failure, allergic reactions, and joint pain.

Yet another class of antidiabetic agents is the gliflozins. These agents act by inhibiting sodium-glucose transport protein 2 (SGLT2) and inhibit reabsorption of glucose in the kidney, thereby lowering blood sugar. These agents include canagliflozin, dapagliflozin, and empagliflozin. Side effects of these agents include urinary tract infections, yeast infections, and ketoacidosis.

Still another class of antidiabetic agents is the glucagon-like peptide-1 receptor agonists. These drugs act by increasing secretion of insulin. These agents include exatenide, liraglutide, lixisenatide, albiglutide, and dulaglutide. These agents may be associated with abnormal pancreatic proliferation.

Yet another class of antidiabetic agents are the amylin analogs, including pram lintide. These drugs are injectable and are intended to be used together with administered insulin.

However, none of the currently approved therapeutic strategies for type 2 diabetes treatment target the underlying problem of reversing insulin resistance by directly enhancing insulin sensitivity.

Epigenetics is broadly described as heritable changes in an organism caused by modifications of gene function that occur without a change in the genetic sequence. Epigenetic regulation of gene expression is a dynamic and reversible process that establishes normal cellular phenotypes but also contributes to human diseases such as Type 2 diabetes. At the molecular level, epigenetic regulation involves hierarchical covalent modification of DNA and the proteins that package DNA, such as histones. These DNA-associated proteins can modulate the expression of DNA. Key protein families that mediate epigenetic signaling through the acetylation and methylation of histones, include histone deacetylases, protein methyltransferases, lysine demethylases, bromodomain-containing proteins and proteins that bind to methylated histones. These protein families are druggable classes of enzymes and druggable classes of protein—protein interaction domains that can be used for therapeutic advantage.

Therefore, there is a need to identify small molecules that target epigenetic enzymes to enhance insulin sensitivity by modifying gene expression in target cells. Such small molecules would target and enhance insulin sensitivity on a molecular basis and would be expected to be freer of side effects than currently used anti-diabetic therapeutic agents.

Additionally, chronic liver disease, such as Nonalcoholic Fatty Liver Disease (NALFD) has been associated with prediabetes and type 2 diabetes and may contribute to elevated blood glucose levels and insulin resistance observed in these disorders. Chronic liver disease can lead to the development of nonalcoholic steatohepatitis, cirrhosis or liver cancer and their related complications. Small molecules that target epigenetic enzymes that can reverse chronic liver disease such as NALFD and that can decrease levels of liver enzymes such as ALT and AST would be of therapeutic advantage.

Additionally, it is now realized that there is a strong association between type 2 diabetes and obesity. Not only is type 2 diabetes far more common in obese individuals than those of normal weight, individuals with type 2 diabetes who were previously obese but who manage to lose enough weight so that they are no longer considered obese have a far better prognosis. Therefore, there is a need to identify small molecules that target epigenetic enzymes and that are associated with obesity management and weight loss.

### SUMMARY OF THE INVENTION

The present invention describes small molecules that have the activity of directly enhancing insulin sensitivity through epigenetic regulation. These small molecules, therefore, provide a new path for the treatment of type 2 diabetes and insulin resistance in type 1 diabetes and also can be used to treat obesity as well as chronic liver disease.

One aspect of the present invention is a method for treatment of type 2 diabetes comprising the step of administering an effective quantity of an epigenetic modulator that modulates expression of at least one gene associated with type 2 diabetes to a subject with type 2 diabetes.

Typically, the epigenetic modulator is selected from the group consisting of a JMJD inhibitor, an HDAC inhibitor, a G9a inhibitor, a SETD7 inhibitor, and a CBP/p300 BRD inhibitor.

When the epigenetic modulator is a JMJD inhibitor, typically the JMJD inhibitor is selected from the group consisting of: 2,4-pyridinedicarboxylic acid; 3-((2-pyridin-2-yl)-6-(1,2,4,5-tetrahydro-3H-benzo[d]azepin-3-yl)pyrimidin-4-yl)amino)propanoic acid; 3-((2-pyridin-3-yl)-6-(1,2,4,5-tetrahydro-3H-benzo[d]azepin-3-yl)pyrimidin-4-yl)amino)propanoic acid; and ethyl 3-((2-pyridin-3-yl)-6-(1,2,4,5-tetrahydro-3H-benzo[d]azepin-3-yl)pyrimidin-4-yl)amino)propanoate. Preferably, the JMJD inhibitor is selected from the group consisting of 2,4-pyridinedicarboxylic acid and 3-((2-pyridin-2-yl)-6-(1,2,4,5-tetrahydro-3H-benzo[d]azepin-3-yl)pyrimidin-4-yl)amino)propanoic acid.

When the epigenetic modulator is an HDAC inhibitor, typically the HDAC inhibitor is selected from the group consisting of: 4-(dimethylamino)-N-(6-hydroxyamino)6-oxohexyl)benzamide; N¹-hydroxy-N⁸-phenyloctanediamide; 4-4-chloro-2-methylphenoxy-N-hydroxybutanamide; pyridine-3-ylmethyl 4-(1-((2-aminophenyl)amino)vinyl)benzyl)carbamate; and 6-(1,3-dioxo-1H-benzo[de]isoquinolin-2(3H)-yl-N-hydroxyhexanamide. Preferably, the HDAC inhibitor is 4-(dimethylamino)-N-(6-hydroxyamino)6-oxohexyl)benzamide.

When the epigenetic modulator is a G9a inhibitor, typically the G9a inhibitor is selected from the group consisting of: 5′-methoxy-6′-(3-pyrrolidin-1-yl)propoxy)spiro[cyclobutane-1,3′indol]-2′-amine; and 7-(2-(2-(dimethylamino)ethoxy)ethoxy)-6-methoxy-2-(4-methyl-1,4-diazepan-1-yl)-N-(1-methylpiperidin-4-yl)quinazolin-4-amine, tri(trifluoroacetate) salt.

When the epigenetic modulator is a SETD7 inhibitor, typically the SETD7 inhibitor is (R)-8-fluoro-N-(1-oxo-1-(pyrrolidin-1-yl)-3-(4-(trifluoromethyl)phenyl)propan-2-yl)-1,2,3,4-tetrahydroisoquinoline-6-sulfonamide.

When the epigenetic modulator is a CBP/p300 BRD inhibitor, typically the a CBP/p300 BRD inhibitor is (S)-4-(1-(2-(3-chloro-4-methoxyphenyl)-6-(3,5-dimethylisoxazol-4-yl)3λ²-benzo[d]imidazole-4-yl)propan-2-yl)morpholine.

In another alternative, the epigenetic modulator is a compound selected from the group consisting of a compound of Formula (I), Formula (II), Formula (III), Formula (IV), Formula (V), Formula (VI), Formula (VII), Formula (VIII), Formula (IX), Formula (X), Formula (XI), Formula (XII), and Formula (XIII) with at least one substituent at a saturated carbon atom selected from the group consisting of C₆-C₁₀ aryl, heteroaryl containing 1-4 heteroatoms selected from N, O, and S, C₁-C₁₀ alkyl, C₁-C₁₀ alkoxy, cycloalkyl, F, amino (NR¹R²), nitro, —SR, —S(O)R, —S(O₂)R, —S(O₂)NR¹R², and —CONR¹R², which can in turn be optionally substituted.

In still another alternative, the epigenetic modulator is a prodrug of a compound selected from the group consisting of a compound of Formula (I), Formula (II), Formula (III), Formula (IV), Formula (V), Formula (VI), Formula (VII), Formula (VIII), Formula (IX), Formula (X), Formula (XI), Formula (XII), and Formula (XIII).

The method described above can further comprise administration of an effective quantity of at least one additional anti-diabetic agent. Typically, the at least one additional anti-diabetic agent is selected from the group consisting of a biguanide, a sulfonylurea, a thiazolidinedione, a DPP-4 inhibitor, a gliflozin, a glucagon-like peptide-1 receptor agonist, and an amylin analog.

Typically, the epigenetic modulator is administered in a pharmaceutical composition comprising: (i) an effective quantity of the epigenetic modulator; and (ii) at least one pharmaceutically acceptable excipient. If at least one additional anti-diabetic agent is administered, in one alternative, the at least one additional anti-diabetic agent is included in the pharmaceutical composition.

Another aspect of the present invention is a pharmaceutical composition for treatment of type 2 diabetes comprising:

(1) an effective quantity of an epigenetic modulator; and

(2) at least one pharmaceutically acceptable excipient.

The epigenetic modulator included in the composition is as described above.

The pharmaceutically acceptable excipient can be selected from the group consisting of:

(1) a preservative;

(2) a sweetening agent;

(3) a thickening agent;

(4) a buffer;

(5) a liquid carrier;

(6) an isotonic agent;

(7) a wetting, solubilizing, or emulsifying agent;

(8) an acidifying agent;

(9) an antioxidant;

(10) an alkalinizing agent;

(11) a carrying agent;

(12) a chelating agent;

(13) a colorant;

(14) a complexing agent;

(15) a solvent;

(16) a suspending and or viscosity-increasing agent;

(17) a flavor or perfume;

(18) an oil;

(19) a penetration enhancer;

(20) a polymer;

(21) a stiffening agent;

(22) a protein;

(23) a carbohydrate;

(24) a bulking agent; and

(25) a lubricating agent.

In one alternative, the composition further comprises an effective quantity of an additional anti-diabetic agent. Typically, the at least one additional anti-diabetic agent is selected from the group consisting of a biguanide, a sulfonylurea, a thiazolidinedione, a DPP-4 inhibitor, a gliflozin, a glucagon-like peptide-1 receptor agonist, and an amylin analog.

Another aspect of the present invention is a prophylactic method for prevention of type 2 diabetes comprising the step of administering an effective quantity of an epigenetic modulator that modulates expression of at least one gene associated with type 2 diabetes to a subject to promote weight loss or weight stabilization and/or reverse chronic liver disease in the subject. The epigenetic modulator is as described above. The method can further comprise administration of an effective quantity of at least one additional anti-diabetic agent; suitable additional anti-diabetic agents are as described above.

Yet another aspect of the present invention is a method for treatment of chronic liver disease comprising the step of administering an effective quantity of an epigenetic modulator that modulates expression of at least one gene associated with type 2 diabetes to a subject with chronic liver disease. The epigenetic modulator is as described above. The method can further comprise administration of an effective quantity of at least one additional agent to treat chronic liver disease. Suitable additional agents to treat chronic liver disease include, but are not limited to, metformin, thiazolidinediones, statins, pentoxyfylline, elafibranor, and obeticholic acid.

### BRIEF DESCRIPTION OF THE DRAWINGS

These and other features, aspects, and advantages of the present invention will become better understood with reference to the following description, appended claims, and accompanying drawings where:

FIG. 1 shows the effect of the compound of Formula (I) on insulin-induced intracellular Ca²⁺ mobilization.

FIG. 2 shows the effect of the compound of Formula (IV) on insulin-induced intracellular Ca²⁺ mobilization.

FIG. 3 shows the effect of the compound of Formula (V) on insulin-induced intracellular Ca²⁺ mobilization.

FIG. 4 shows the effect of the compound of Formula (VI) on insulin-induced intracellular Ca²⁺ mobilization.

FIG. 5 shows results for the compound of Formula (I) after 11 days of treatment in the mouse model of severe type 2 diabetes and obesity in terms of decrease in fed and fasting glucose levels.

FIG. 6 shows results for the compound of Formula (I) after 29 days of treatment in the mouse model of severe type 2 diabetes and obesity in terms of enhancing glucose clearance in a glucose tolerance test (top panel, blood glucose in mg/dL plotted versus time after glucose load in minutes; bottom panel, total area under the curve for blood glucose).

FIG. 7 shows results in terms of blood glucose levels for the compound of Formula (II) after 19 days (top panel) or 29-30 days (bottom panel).

FIG. 8 shows results in terms of decrease of body weight for the compound of Formula (III) was found to significantly decrease body weight after 9 days of treatment in the mouse model of severe type 2 diabetes and obesity.

FIG. 9 is a summary chart showing results with 2,4-pyridinedicarboxylic acid (Formula I), SAHA (Formula (IV)), A-366 (Formula (V)), R-PFI-2 (Formula (VI)), and SGC-CBP30 (Formula (VII)) showing the percentage increase of insulin mediated signaling after 24 hours and 48 hours.

FIG. 10 is a summary chart showing the results with GSK-J2 (Formula (VIII)), GSK-J5 (Formula (IX)), droxinostat (Formula (X)), entinostat (Formula (XI)), scriptaid (Formula (XII)), CAY10398 (Formula (III), and UNC0321 (Formula (XIII)) showing the percentage increase of insulin mediated signaling after 24 hours and 48 hours.

FIG. 11 is a graph showing that the compound of Formula (I) significantly lowers enzymes (ALT and AST) associated with liver disease measured in blood serum by 68% in a mouse model of Type 2 diabetes and severe obesity.

### DETAILED DESCRIPTION OF THE INVENTION

The present application describes and utilizes a strategy to identify small molecules that target epigenetic enzymes to enhance insulin sensitivity by modifying gene expression in target cells.

The following terms, among others, are used to describe the present invention. It is to be understood that a term which is not specifically defined is to be given a meaning consistent with the use of that term within the context of the present invention as understood by those of ordinary skill.

As used herein, the term “compound,” as used herein, unless otherwise indicated, refers to any specific chemical compound disclosed herein and includes tautomers, regioisomers, geometric isomers, and where applicable, optical isomers (e.g. enantiomers or diastereomers) thereof, as well as pharmaceutically acceptable salts and derivatives (including prodrug forms) thereof. Within its use in context, the term compound generally refers to a single compound, but also may include other compounds such as stereoisomers, regioisomers and/or optical isomers (including racemic mixtures) as well as specific enantiomers or enantiomerically enriched mixtures of disclosed compounds as well as diastereomers and epimers, where applicable in context. The term also refers, in context to prodrug forms of compounds which have been modified to facilitate the administration and delivery of compounds to a site of activity.

As used herein, the terms “patient” or “subject” are used throughout the specification within context to describe an animal, generally a mammal and preferably a human, to whom treatment, including prophylactic treatment (prophylaxis), with the methods or compositions according to the present invention is provided. As used herein, the term “effective” is used herein, unless otherwise indicated, to describe an amount of a compound or composition which, in context, is used to produce or effect an intended result as described herein with respect to type 2 diabetes, and, where appropriate, with respect to type 1 diabetes or prediabetes or obesity and chronic liver disease. This term subsumes all other effective amount or effective concentration terms (including the term “therapeutically effective”) which are otherwise described in the present application.

As used herein, the term “pharmaceutically acceptable salt” or “salt” is used throughout the specification to describe a salt form of one or more of the compositions herein which are presented to increase the solubility of the compound in saline for parenteral delivery or in the gastric juices of the patient's gastrointestinal tract in order to promote dissolution and the bioavailability of the compounds. Pharmaceutically acceptable salts include those derived from pharmaceutically acceptable inorganic or organic bases and acids. Suitable salts include those derived from alkali metals such as potassium and sodium, alkaline earth metals such as calcium, magnesium and ammonium salts, among numerous other acids well known in the pharmaceutical art. Sodium and potassium salts may be preferred as neutralization salts of carboxylic acids and free acid phosphate containing compositions according to the present invention. The term “salt” shall mean any salt consistent with the use of the compounds according to the present invention unless a specific salt or salts are specified. In the case where the compounds are used in pharmaceutical indications, including the treatment of type 2 diabetes, and, where appropriate, type 1 diabetes or prediabetes or obesity or chronic liver disease, the term “salt” shall mean a pharmaceutically acceptable salt, consistent with the use of the compounds as pharmaceutical agents.

As used herein, the term “co-administration” shall mean that at least two compounds or compositions are administered to the patient at the same time, such that effective amounts or concentrations of each of the two or more compounds may be found in the patient at a given point in time. Although compounds according to the present invention may be co-administered to a patient at the same time, the term embraces both administration of two or more agents at the same time or at different times, including sequential administration. Preferably, effective concentrations of all co-administered compounds or compositions are found in the subject at a given time.

As used herein, the term “diabetes,” without further limitation, refers to type 2 diabetes. However, certain methods and compositions according to the present invention may be useful for the treatment of type 1 diabetes or prediabetes or obesity or chronic liver disease as well. The recitation of “type 2 diabetes” in the present application is not to be interpreted to mean that any method or composition recited in the present application is not useful for the treatment of type 1 diabetes or prediabetes or obesity or chronic liver disease.

As used herein, the term “obesity” refers to a body mass index (BMI) of greater than 30.0. In general, a BMI from 30.0 to 35.0 is defined as class I obesity. A BMI from 35.0 to 40.0 is defined as class II obesity. A BMI of over 40.0 is defined as class III obesity. Obesity is associated with an increased risk of cardiovascular disease, hypertension, type 2 diabetes, sleep apnea, certain types of cancer, osteoarthritis and asthma, and may aggravate musculoskeletal conditions such as those resulting in back pain.

As used herein, the terms “treating,” treatment” or similar terminology include any improvement or reduction of progression of type 2 diabetes, or, where appropriate, type 1 diabetes or prediabetes or obesity or chronic liver disease, which can be evaluated by one or more of the following criteria: reduction in blood glucose; reduction in glycated hemoglobin; improvement in response to glucose tolerance test; reduction in urinary frequency, urinary urgency, or excessive thirst; reduction in pain associated with peripheral neuropathy; improvement in wound healing, improvement in fatigue, body mass index, liver enzyme levels or any other sign or symptom associated with type 2 diabetes. The terms “treating,” treatment” or similar terminology are not intended to imply a permanent cure for type 2 diabetes, or, where appropriate, type 1 diabetes or prediabetes or chronic liver disease. Compositions and methods according to the present invention are not limited to treatment of humans, but are applicable to treatment of socially or economically important animals, such as dogs, cats, horses, cows, sheep, goats, pigs, and other animal species of social or economic importance. Unless specifically stated, compositions and methods according to the present invention are not limited to the treatment of humans.

Additional definitions are provided below with respect to substitutions that can be made in therapeutically active agents according to the present invention.

An epigenetic screen of a library of small molecules was performed as follows. L6 rat myoblasts (skeletal muscle cells) were incubated with potential epigenetic compounds from a large library of small molecules (1 μM) for either 24 or 48 hours. The extent of insulin-mediated intracellular Ca²⁺ release in the L6 rat myoblasts was then measured in a 384-well format using a FLIPR high-throughput cellular screening system (Molecular Devices, Sunnyvale, Calif.). Real-time kinetics of intracellular Ca²⁺ mobilization were recorded for 30 min following insulin stimulation of cells.

Several classes of epigenetic modulators were identified: (1) JMJD inhibitors (lysine demethylase inhibitors); (2) HDAC inhibitors (histone deacetylase inhibitors); (3) G9a inhibitors (lysine methyltransferase inhibitors); (4) SETD7 inhibitors (lysine methyltransferase inhibitors); and (5) CBP/p300 BRD inhibitors (histone acetyltransferase/bromodomain inhibitors).

Lysine demethylase catalyzes the removal of methyl groups from the N6-position of lysines, particularly in histones. The enzyme can catalyze the removal of one or two methyl groups from the N6-position of lysines, and so can convert a dimethylated lysine residue into a monomethylated lysine residue or a lysine residue with no methyl groups.

Histone deacetylase catalyzes the removal of acetyl groups from an ε-N-acetyllysine amino acid located in a histone molecule. The removal of the acetyl groups increases the binding affinity of the histones for the DNA molecules to which they bind.

Lysine methyltransferase catalyzes the transfer of one, two, or three methyl groups to lysine residues in proteins, particularly in histones.

Histone acetyltransferase catalyzes the addition of acetyl groups to the 8-amino group of lysine residues in histone molecules to create. This may render DNA more accessible to transcription factors by reducing the affinity of the histone molecules for the DNA; additionally, the formation of ε-N-acetyllysine may result in the generation of binding sites for specific protein-protein interaction domains, such as the ε-N-acetyllysine-binding bromodomain.

The following small molecule epigenetic modulators have been identified that can be used to treat type 2 diabetes.

The first of these compounds is 2,4-pyridinedicarboxylic acid, shown below as Formula (I):

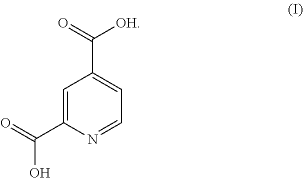 [SMILES: O=C(O)c1ccnc(C(=O)O)c1]

The compound of Formula (I) acts as a JMJD inhibitor. JMJD is a lysine-specific demethylase.

The second of these compounds is GSK-J1 (3-((2-pyridin-2-yl)-6-(1,2,4,5-tetrahydro-3H-benzo[d]azepin-3-yl)pyrimidin-4-yl)amino)propanoic acid), shown below as Formula (II):

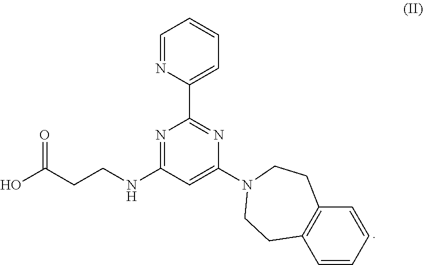 [SMILES: O=C(O)CCNc1cc(N2CCc3ccccc3CC2)nc(-c2ccccn2)n1]

The compound of Formula (II) is also a JMJD inhibitor.

The third of these compounds is CAY10398 (4-(dimethylamino)-N-(6-hydroxyamino)6-oxohexyl)benzamide), shown below as Formula (III):

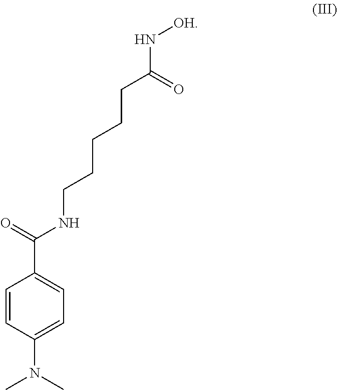 [SMILES: CN(C)c1ccc(C(=O)NCCCCCC(=O)NO)cc1]

The compound of Formula (III) is an HDAC inhibitor.

Other compounds affecting enzymes associated with epigenetic modification and that can be used to treat diabetes include the following.

One of the additional compounds is SAHA (N¹-hydroxy-N⁸-phenyloctanediamide), shown below as Formula (IV):

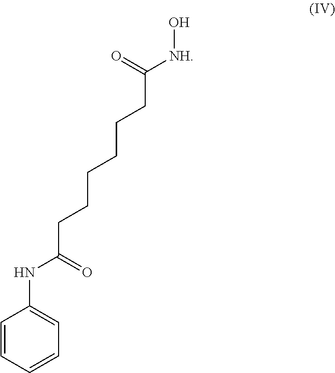 [SMILES: O=C(CCCCCCC(=O)Nc1ccccc1)NO]

The compound of Formula (IV) is an HDAC inhibitor.

Another of the additional compounds is A-366 (5′-methoxy-6′-(3-pyrrolidin-1-yl)propoxy)spiro[cyclobutane-1,3′indol]-2′-amine), shown below as Formula (V):

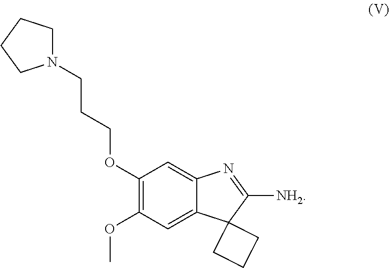 [SMILES: COc1cc2c(cc1OCCCN1CCCC1)N=C(N)C21CCC1]

The compound of Formula (V) is a G9a inhibitor. The enzyme G9a is a lysine methyltransferase.

Yet another of the additional compounds is (R)PFI-2 ((R)-8-fluoro-N-(1-oxo-1-(pyrrolidin-1-yl)-3-(4-(trifluoromethyl)phenyl)propan-2-yl)-1,2,3,4-tetrahydroisoquinoline-6-sulfonamide), shown below as Formula (VI):

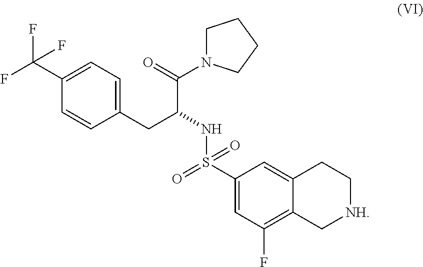 [SMILES: O=C([C@@H](Cc1ccc(C(F)(F)F)cc1)NS(=O)(=O)c1cc(F)c2c(c1)CCNC2)N1CCCC1]

The compound of Formula (VI) is a SETD7 inhibitor. SETD7 is a lysine methyltransferase.

Yet another of the additional compounds is SGC-CBP30 ((S)-4-(1-(2-(3-chloro-4-methoxyphenyl)-6-(3,5-dimethylisoxazol-4-yl)3λ²-benzo[d]imidazole-4-yl)propan-2-yl)morpholine), shown below as Formula (VII):

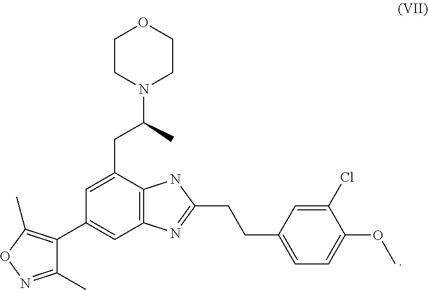 [SMILES: COc1ccc(CCc2nc3cc(-c4c(C)noc4C)cc(C[C@H](C)N4CCOCC4)c3[nH]2)cc1Cl]

The compound of Formula (VII) is a histone acetyltransferase-bromodomain inhibitor.

Still other additional compounds affecting enzymes associated with epigenetic modification and that can be used to treat diabetes include the following.

Another of the additional compounds is GSK-J2 (3-((2-pyridin-3-yl)-6-(1,2,4,5-tetrahydro-3H-benzo[d]azepin-3-yl)pyrimidin-4-yl)amino)propanoic acid), shown below as Formula (VIII):

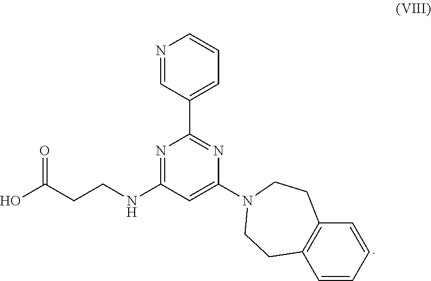 [SMILES: O=C(O)CCNc1cc(N2CCc3ccccc3CC2)nc(-c2cccnc2)n1]

The compound of Formula (VIII) is a JMJD inhibitor.

Yet another of the additional compounds is GSK-J5 (ethyl 3-((2-pyridin-3-yl)-6-(1,2,4,5-tetrahydro-3H-benzo[d]azepin-3-yl)pyrimidin-4-yl)amino)propanoate), shown below as Formula (IX):

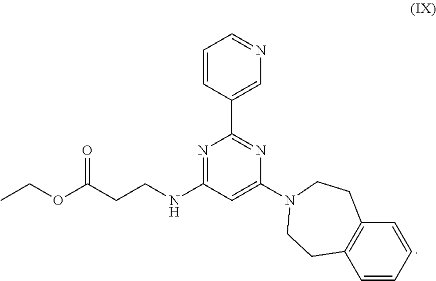 [SMILES: CCOC(=O)CCNc1cc(N2CCc3ccccc3CC2)nc(-c2cccnc2)n1]

The compound of Formula (IX) is a JMJD inhibitor.

Yet another of the additional compounds is droxinostat (4-4-chloro-2-methylphenoxy-N-hydroxybutanamide), shown below as Formula (X):

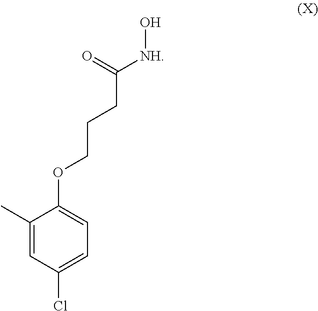 [SMILES: Cc1cc(Cl)ccc1OCCCC(=O)NO]

The compound of Formula (X) is a HDAC inhibitor.

Yet another of the additional compounds is entinostat (pyridine-3-ylmethyl 4-(1-((2-aminophenyl)amino)vinyl)benzyl)carbamate), shown below as Formula (XI):

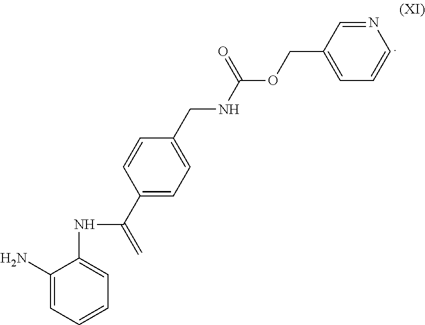 [SMILES: C=C(Nc1ccccc1N)c1ccc(CNC(=O)OCc2cccnc2)cc1]

The compound of Formula (XI) is a HDAC inhibitor.

Yet another of the additional compounds is scriptaid (6-(1,3-dioxo-1H-benzo[de]isoquinolin-2(3H)-yl-N-hydroxyhexanamide), shown below as Formula (XII):

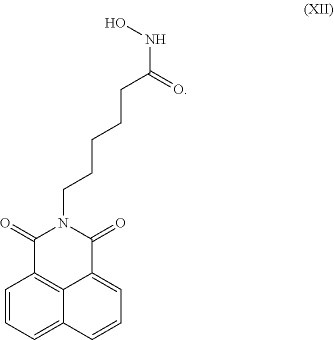 [SMILES: O=C(CCCCCN1C(=O)c2cccc3cccc(c23)C1=O)NO]

The compound of Formula (XII) is a HDAC inhibitor.

Still another of the additional compounds is UNC0321 (7-(2-(2-(dimethylamino)ethoxy)ethoxy)-6-methoxy-2-(4-methyl-1,4-diazepan-1-yl)-N-(1-methylpiperidin-4-yl)quinazolin-4-amine, tri(trifluoroacetate) salt), shown below as Formula (XIII):

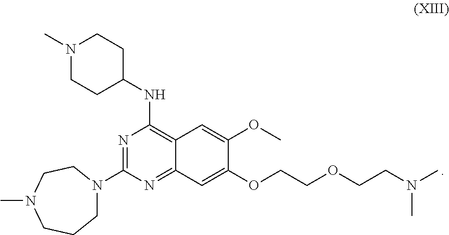

The compound of Formula (XIII) is a G9a inhibitor.

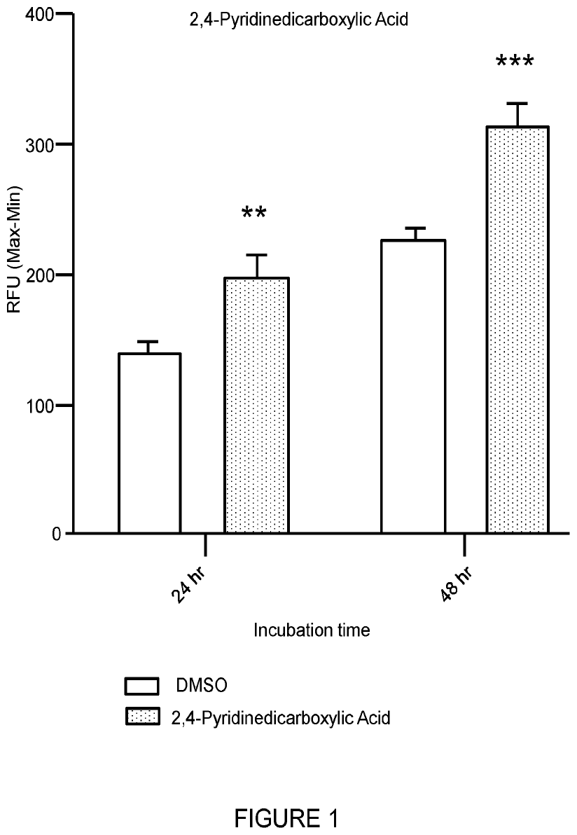

FIG. 1 shows the effect of the compound of Formula (I) on insulin-induced intracellular Ca²⁺ mobilization. (In FIGS. 1-4, the term “RFU” means relative fluorescence units.)

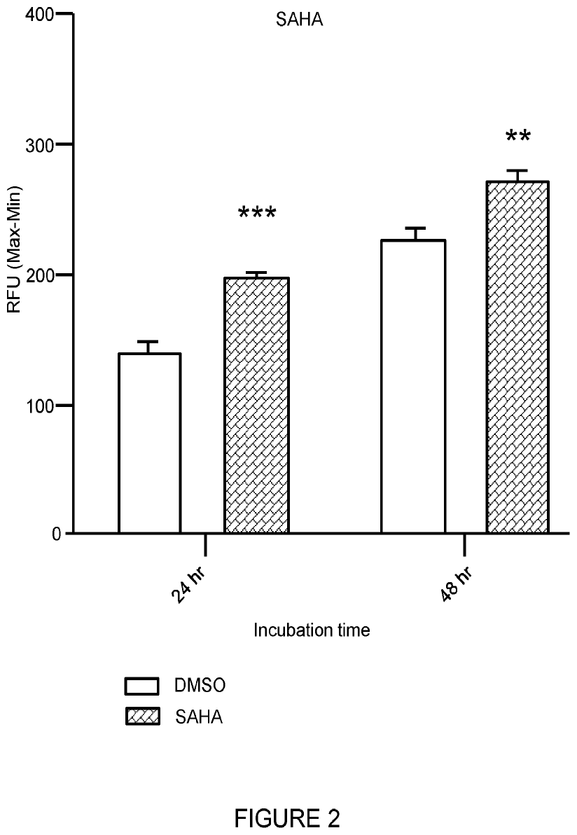

FIG. 2 shows the effect of the compound of Formula (IV) on insulin-induced intracellular Ca²⁺ mobilization.

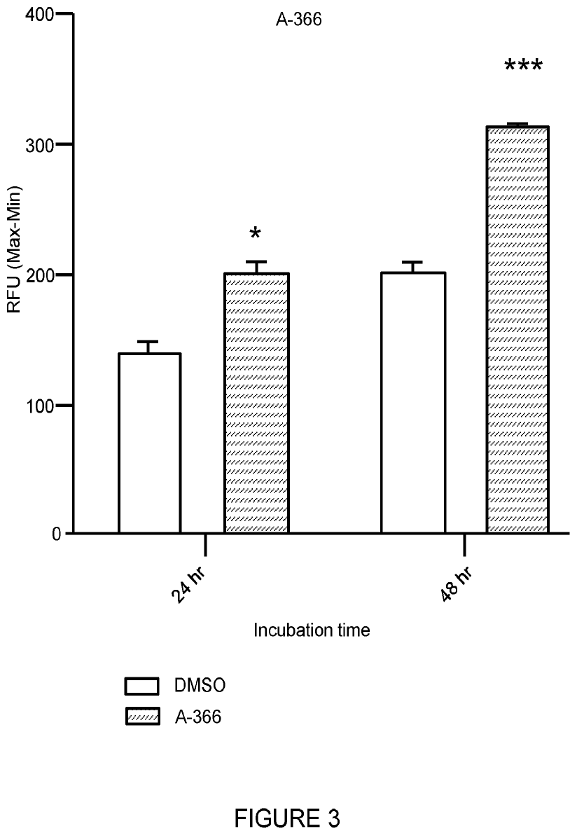

FIG. 3 shows the effect of the compound of Formula (V) on insulin-induced intracellular Ca²⁺ mobilization.

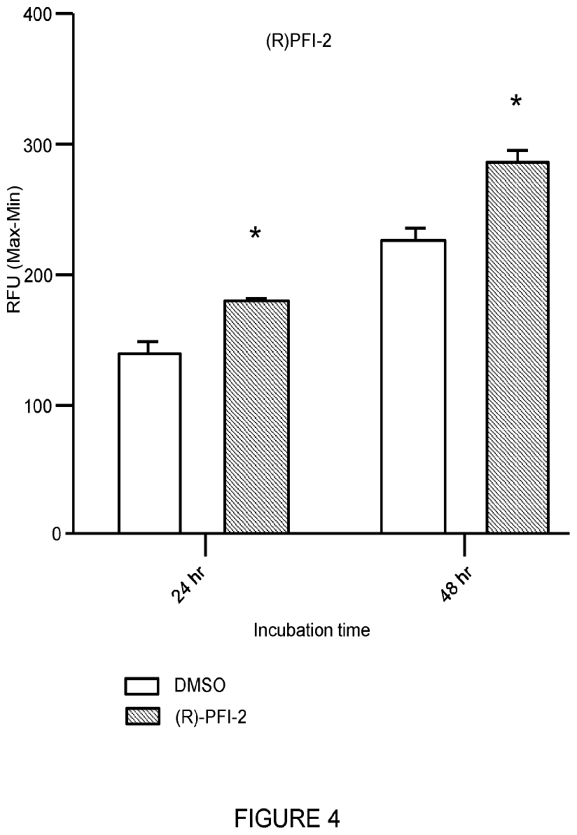

FIG. 4 shows the effect of the compound of Formula (VI) on insulin-induced intracellular Ca²⁺ mobilization.

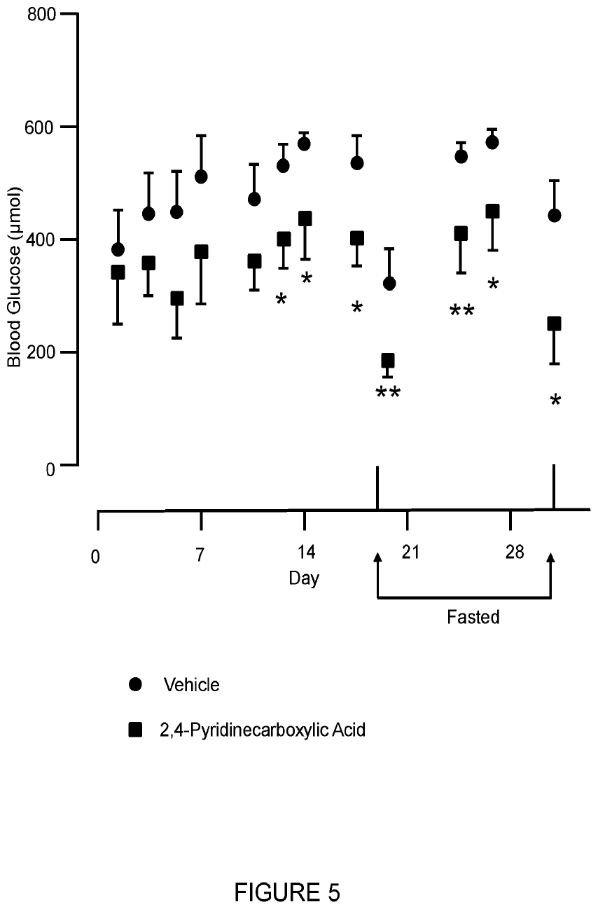

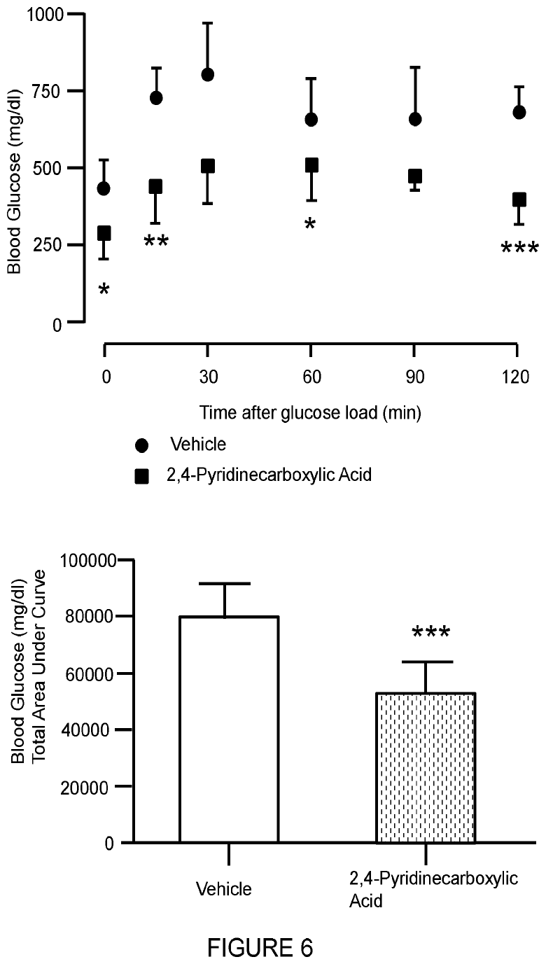

Additionally, JMJD inhibitors, including compounds of Formula (I) and Formula (II) were found to significantly decrease blood glucose levels in a mouse model of severe type 2 diabetes and obesity. The results for the compound of Formula (I) after 11 days of treatment in the mouse model of severe type 2 diabetes and obesity are shown in FIG. 5. The compound of Formula (I) was also shown to significantly enhance glucose clearance using the glucose tolerance test in the mouse model of severe type 2 diabetes and obesity; the results are shown in FIG. 6.

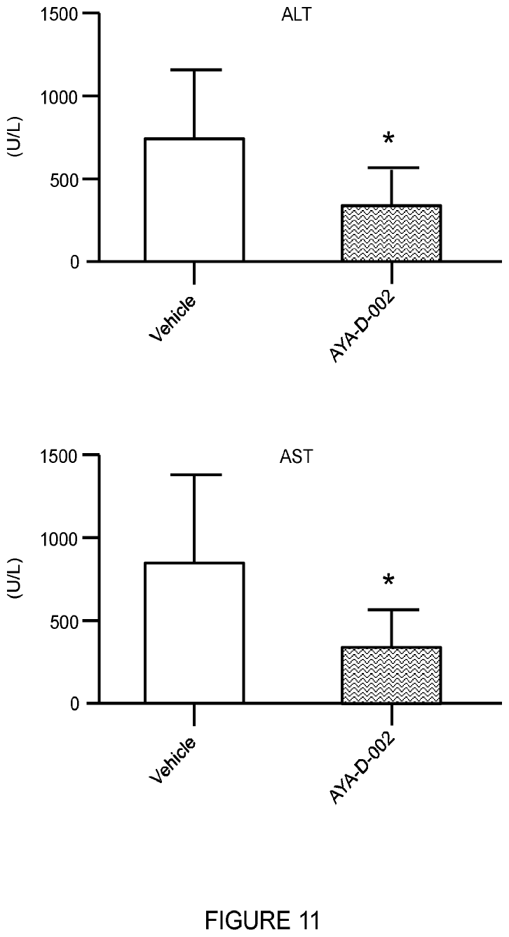

The results in terms of liver enzyme levels for the compound of Formula (I) after 29-30 days are shown in FIG. 11. FIG. 11 shows that the compound of Formula (I) significantly lowers enzymes (ALT and AST) associated with liver disease measured in blood serum by 68% in a mouse model of Type 2 diabetes and severe obesity.

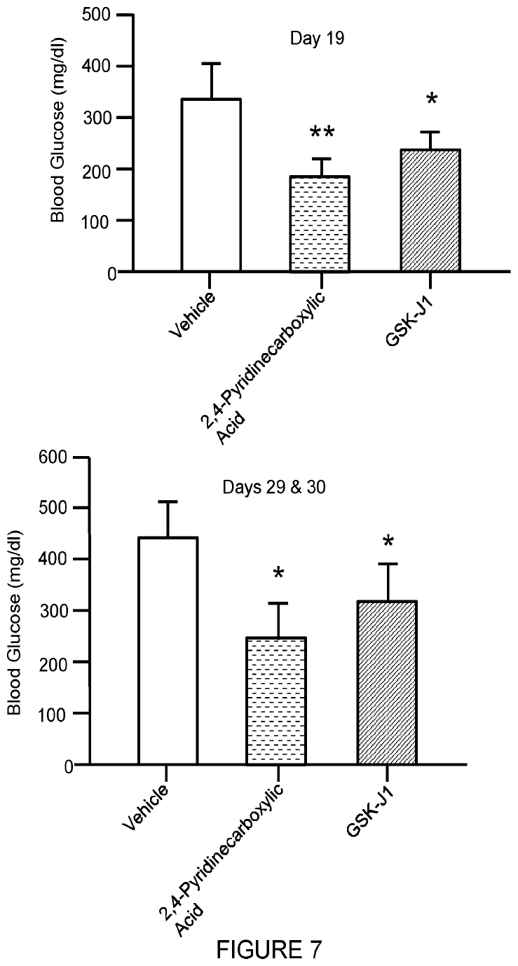

The results in terms of blood glucose levels for the compound of Formula (II) after 19 days (top panel) or 29-30 days (bottom panel) are shown in FIG. 7.

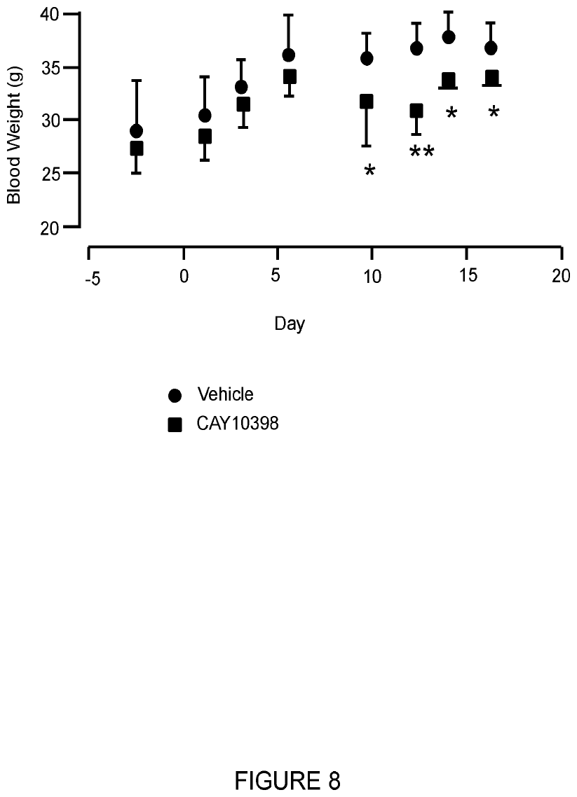

The compound of Formula (III) was found to significantly decrease body weight after 9 days of treatment in the mouse model of severe type 2 diabetes and obesity; the results are shown in FIG. 8.

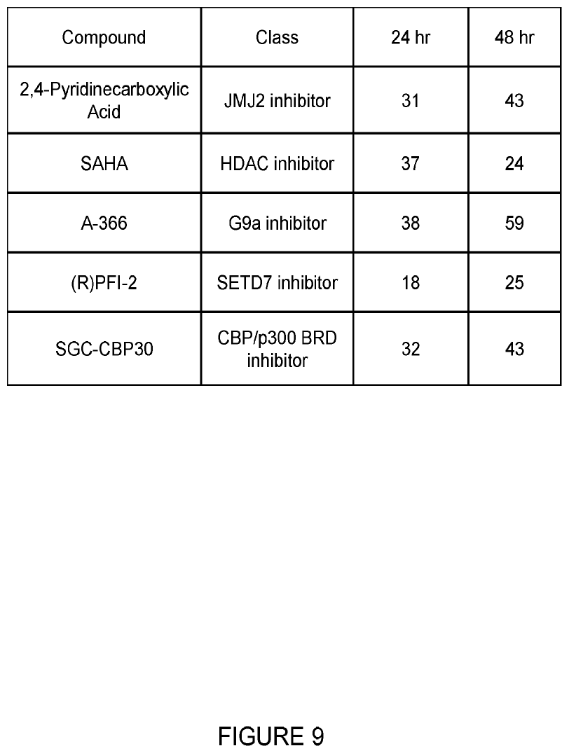

A summary chart of the results with 2,4-pyridinedicarboxylic acid (Formula I), SAHA (Formula (IV)), A-366 (Formula (V)), R-PFI-2 (Formula (VI)), and SGC-CBP30 (Formula (VII)) showing the percentage increase of insulin mediated signaling after 24 hours and 48 hours is shown in FIG. 9.

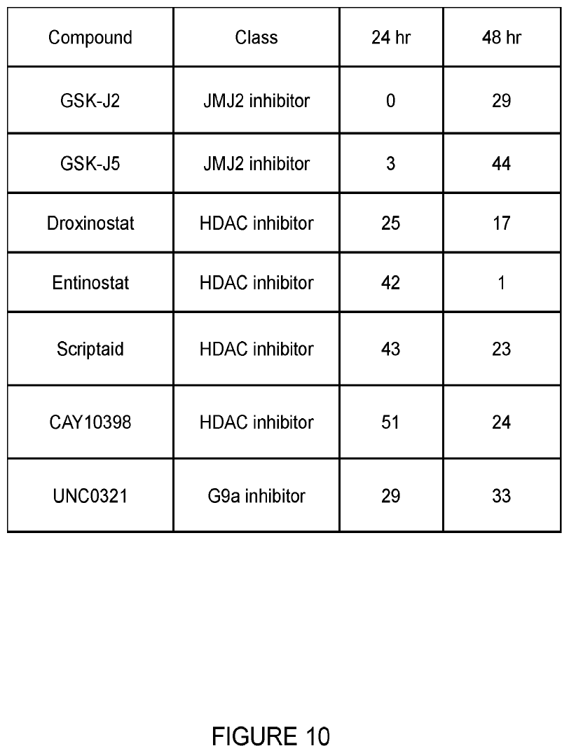

A summary chart of the results with GSK-J2 (Formula (VIII)), GSK-J5 (Formula (IX)), droxinostat (Formula (X)), entinostat (Formula (XI)), scriptaid (Formula (XII)), CAY10398 (Formula (III), and UNC0321 (Formula (XIII)) showing the percentage increase of insulin mediated signaling after 24 hours and 48 hours is shown in FIG. 10.

In in vivo testing for efficacy in a mouse model of type 2 diabetes, the compounds as described above were tested for efficacy in a leptin receptor deficient transgenic mouse model (db/db) that exhibits severe obesity and Type 2 diabetes. Fed blood glucose levels were measured over a period of 28 days. Fasting glucose levels were measured on days 19, 29, and 30. Glucose clearance was measured using the glucose tolerance test on days 29 and 30. Body weight was also measured as compounds may regulate the expression of genes that are involved in obesity.

Accordingly, one aspect of the invention is a method for treating type 2 diabetes by administering an effective quantity of an epigenetic modulator that modulates expression of at least one gene associated with type 2 diabetes to a subject with type 2 diabetes.

Typically, the epigenetic modulator is selected from the group consisting of a JMJD inhibitor, an HDAC inhibitor, a G9a inhibitor, a SETD7 inhibitor, and a CBP/p300 BRD inhibitor.

When the epigenetic modulator is a JMJD inhibitor, typically the JMJD inhibitor is a compound selected from the group consisting of Formula (I), Formula (II), Formula (VIII), and Formula (IX). Preferably, when the epigenetic modulator is a JMJD inhibitor, the JMJD inhibitor is a compound selected from the group consisting of Formula (I) and Formula (II).

Typically, when the epigenetic modulator is a HDAC inhibitor, the HDAC inhibitor is a compound selected from the group consisting of Formula (III), Formula (IV), Formula (X), Formula (XI), and Formula (XII). Preferably, when the epigenetic modulator is a HDAC inhibitor, the HDAC inhibitor is a compound of Formula (III).

Typically, when the epigenetic modulator is a G9a inhibitor, the G9a inhibitor is a compound selected from the group consisting of Formula (V) and Formula (XIII).

When the epigenetic modulator is a SETD7 inhibitor, the SETD7 inhibitor is typically Formula (VI).

When the epigenetic modulator is a CBP/p300 BRD inhibitor, the CBP/p300 BRD inhibitor is typically Formula (VII).

Also within the scope of the inventions are derivatives of compounds of Formula (I), Formula (II), Formula (III), Formula (IV), Formula (V), Formula (VI), Formula (VII), Formula (VIII), Formula (IX), Formula (X), Formula (XI), Formula (XII), and Formula (XIII) that have one or more optional substituents. In general, for optional substituents at saturated carbon atoms such as those that are part of the structures of Formula (I), Formula (II), Formula (III), Formula (IV), Formula (V), Formula (VI), Formula (VII), Formula (VIII), Formula (IX), Formula (X), Formula (XI), Formula (XII), and Formula (XIII), the following substituents can be employed: C₆-C₁₀ aryl, heteroaryl containing 1-4 heteroatoms selected from N, O, and S, C₁-C₁₀ alkyl, C₁-C₁₀ alkoxy, cycloalkyl, F, amino (NR¹R²), nitro, —SR, —S(O)R, —S(O₂)R, —S(O₂)NR¹R², and —CONR¹R², which can in turn be optionally substituted. Further descriptions of potential optional substituents are provided below. Additional optional substituents are also further described below.

Optional substituents as described above that are within the scope of the present invention do not substantially affect the activity of the derivative or the stability of the derivative, particularly the stability of the derivative in aqueous solution or the bioavailability of the derivative when administered orally. Definitions for a number of common groups that can be used as optional substituents are provided below; however, the omission of any group from these definitions cannot be taken to mean that such a group cannot be used as an optional substituent as long as the chemical and pharmacological requirements for an optional substituent are satisfied.

As used herein, the term “alkyl” refers to an unbranched, branched, or cyclic saturated hydrocarbyl residue, or a combination thereof, of from 1 to 12 carbon atoms that can be optionally substituted; the alkyl residues contain only C and H when unsubstituted. Typically, the unbranched or branched saturated hydrocarbyl residue is from 1 to 6 carbon atoms, which is referred to herein as “lower alkyl.” When the alkyl residue is cyclic and includes a ring, it is understood that the hydrocarbyl residue includes at least three carbon atoms, which is the minimum number to form a ring; such alkyl groups are referred to generally as “cycloalkyl.” An alkyl residue, including a cycloalkyl residue, may itself be further substituted with further alkyl, cycloalkyl, or other groups. As used herein, the term “alkenyl” refers to an unbranched, branched or cyclic hydrocarbyl residue having one or more carbon-carbon double bonds. As used herein, the term “alkynyl” refers to an unbranched, branched, or cyclic hydrocarbyl residue having one or more carbon-carbon triple bonds; the residue can also include one or more double bonds. With respect to the use of “alkenyl” or “alkynyl,” the presence of multiple double bonds cannot produce an aromatic ring. As used herein, the terms “hydroxyalkyl,” “hydroxyalkenyl,” and “hydroxyalkynyl,” respectively, refer to an alkyl, alkenyl, or alkynyl group including one or more hydroxyl groups as substituents; as detailed below, further substituents can be optionally included. As used herein, the term “aryl” refers to a monocyclic or fused bicyclic moiety having the well-known characteristics of aromaticity; examples include phenyl, naphthyl, fluorenyl, and indenyl, which can be optionally substituted. As used herein, the term “hydroxyaryl” refers to an aryl group including one or more hydroxyl groups as substituents; as further detailed below, further substituents can be optionally included. As used herein, the term “heteroaryl” refers to monocyclic or fused bicylic ring systems that have the characteristics of aromaticity and include one or more heteroatoms selected from 0, S, and N. The inclusion of a heteroatom permits aromaticity in 5-membered rings as well as in 6-membered rings. Typical heteroaromatic systems include monocyclic C₅-C₆ heteroaromatic groups such as pyridyl, pyrimidyl, pyrazinyl, thienyl, furanyl, pyrrolyl, pyrazolyl, thiazolyl, oxazolyl, triazolyl, triazinyl, tetrazolyl, tetrazinyl, and imidazolyl, as well as the fused bicyclic moieties formed by fusing one of these monocyclic heteroaromatic groups with a phenyl ring or with any of the heteroaromatic monocyclic groups to form a C₈-C₁₀ bicyclic group such as indolyl, benzimidazolyl, indazolyl, benzotriazolyl, isoquinolyl, quinolyl, benzothiazolyl, benzofuranyl, pyrazolylpyridyl, quinazolinyl, quinoxalinyl, cinnolinyl, and other ring systems known in the art. Any monocyclic or fused ring bicyclic system that has the characteristics of aromaticity in terms of delocalized electron distribution throughout the ring system is included in this definition. This definition also includes bicyclic groups where at least the ring that is directly attached to the remainder of the molecule has the characteristics of aromaticity, including the delocalized electron distribution that is characteristic of aromaticity. Typically the ring systems contain 5 to 12 ring member atoms and up to four heteroatoms, wherein the heteroatoms are selected from the group consisting of N, O, and S. Frequently, the monocyclic heteroaryls contain 5 to 6 ring members and up to three heteroatoms selected from the group consisting of N, O, and S; frequently, the bicyclic heteroaryls contain 8 to 10 ring members and up to four heteroatoms selected from the group consisting of N, O, and S. The number and placement of heteroatoms in heteroaryl ring structures is in accordance with the well-known limitations of aromaticity and stability, where stability requires the heteroaromatic group to be stable enough to be exposed to water at physiological temperatures without rapid degradation. As used herein, the term “hydroxheteroaryl” refers to a heteroaryl group including one or more hydroxyl groups as substituents; as further detailed below, further substituents can be optionally included. As used herein, the terms “haloaryl” and “haloheteroaryl” refer to aryl and heteroaryl groups, respectively, substituted with at least one halo group, where “halo” refers to a halogen selected from the group consisting of fluorine, chlorine, bromine, and iodine, typically, the halogen is selected from the group consisting of chlorine, bromine, and iodine; as detailed below, further substituents can be optionally included. As used herein, the terms “haloalkyl,” “haloalkenyl,” and “haloalkynyl” refer to alkyl, alkenyl, and alkynyl groups, respectively, substituted with at least one halo group, where “halo” refers to a halogen selected from the group consisting of fluorine, chlorine, bromine, and iodine, typically, the halogen is selected from the group consisting of chlorine, bromine, and iodine; as detailed below, further substituents can be optionally included.

As used herein, the term “optionally substituted” indicates that the particular group or groups referred to as optionally substituted may have no non-hydrogen substituents, or the group or groups may have one or more non-hydrogen substituents consistent with the chemistry and pharmacological activity of the resulting molecule. If not otherwise specified, the total number of such substituents that may be present is equal to the total number of hydrogen atoms present on the unsubstituted form of the group being described; fewer than the maximum number of such substituents may be present. Where an optional substituent is attached via a double bond, such as a carbonyl oxygen (C═O), the group takes up two available valences on the carbon atom to which the optional substituent is attached, so the total number of substituents that may be included is reduced according to the number of available valences. As used herein, the term “substituted,” whether used as part of “optionally substituted” or otherwise, when used to modify a specific group, moiety, or radical, means that one or more hydrogen atoms are, each, independently of each other, replaced with the same or different substituent or substituents.

Substituent groups useful for substituting saturated carbon atoms in the specified group, moiety, or radical include, but are not limited to, —Z^{a}, ═O, —OZ^{b}, —SZ^{b}, ═S⁻, —NZ^{c}Z^{c}, ═NZ^{b}, ═N—OZ^{b}, trihalomethyl, —CF₃, —CN, —OCN, —SCN, —NO, —NO₂, ═N₂, —N₃, —S(O)₂Z^{b}, —S(O)₂NZ^{b}, —S(O₂)O⁻, —S(O₂)OZ^{b}, —OS(O₂)OZ^{b}, —OS(O₂)O⁻, —OS(O₂)OZ^{b}, —P(O)(O⁻)₂, —P(O)(OZ^{b})(O⁻), —P(O)(OZ^{b})(OZ^{b}), —C(O)Z^{b}, —C(S)Z^{b}, —C(NZ^{b})Z^{b}, —C(O)O⁻, —C(O)OZ^{b}, —C(S)OZ^{b}, —C(O)NZ^{c}Z^{c}, —C(NZ^{b})NZ^{c}Z^{c}, —OC(O)Z^{b}, —OC(S)Z^{b}, —OC(O)O⁻, —OC(O)OZ^{b}, —OC(S)OZ^{b}, —NZ^{b}C(O)Z^{b}, —NZ^{b}C(S)Z^{b}, —NZ^{b}C(O)O⁻, —NZ^{b}C(O)OZ^{b}, —NZ^{b}C(S)OZ^{b}, —NZ^{b}C(O)NZ^{c}Z^{c}, —NZ^{b}C(NZ^{b})Z^{b}, —NZ^{b}C(NZ^{b})NZ^{c}Z^{c}, wherein Z^{a} is selected from the group consisting of alkyl, cycloalkyl, heteroalkyl, cycloheteroalkyl, aryl, arylalkyl, heteroaryl and heteroarylalkyl; each Z^{b} is independently hydrogen or Z^{a}; and each Z^{c} is independently Z^{b} or, alternatively, the two Z^{c}'s may be taken together with the nitrogen atom to which they are bonded to form a 4-, 5-, 6-, or 7-membered cycloheteroalkyl ring structure which may optionally include from 1 to 4 of the same or different heteroatoms selected from the group consisting of N, O, and S. As specific examples, —NZ^{c}Z^{c} is meant to include —NH₂, —NH-alkyl, —N-pyrrolidinyl, and —N-morpholinyl, but is not limited to those specific alternatives and includes other alternatives known in the art. Similarly, as another specific example, a substituted alkyl is meant to include -alkylene-O-alkyl, -alkylene-heteroaryl, -alkylene-cycloheteroaryl, -alkylene-C(O)OZ^{b}, -alkylene-C(O)NZ^{b}Z^{b}, and —CH₂—CH₂—C(O)—CH₃, but is not limited to those specific alternatives and includes other alternatives known in the art. The one or more substituent groups, together with the atoms to which they are bonded, may form a cyclic ring, including, but not limited to, cycloalkyl and cycloheteroalkyl.

Similarly, substituent groups useful for substituting unsaturated carbon atoms in the specified group, moiety, or radical include, but are not limited to, —Z^{a}, halo, —O⁻, —OZ^{b}, —SZ^{b}, —S⁻, —NZ^{c}Z^{c}, trihalomethyl, —CF₃, —CN, —OCN, —SCN, —NO, —NO₂, —N₃, —S(O)₂Z^{b}, —S(O₂)O⁻, —S(O₂)OZ^{b}, —OS(O₂)OZ^{b}, —OS(O₂)O⁻, —P(O)(O⁻)₂, —P(O)(OZ^{b})(O⁻), —P(O)(OZ^{b})(OZ^{b}), —C(O)Z^{b}, —C(S)Z^{b}, —C(NZ^{b})Z^{b}, —C(O)O⁻, —C(O)OZ^{b}, —C(S)OZ^{b}, —C(O)NZ^{c}Z^{c}, —C(NZ^{b})NZ^{c}Z^{c}, —OC(O)Z^{b}, —OC(S)Z^{b}, —OC(O)O⁻, —OC(O)OZ^{b}, —OC(S)OZ^{b}, —NZ^{b}C(O)OZ^{b}, —NZ^{b}C(S)OZ^{b}, —NZ^{b}C(O)NZ^{c}Z^{c}, —NZ^{b}C(NZ^{b})Z^{b}, and —NZ^{b}C(NZ^{b})NZ^{c}Z^{c}, wherein Z^{a}, Z^{b}, and Z^{c} are as defined above.

Similarly, substituent groups useful for substituting nitrogen atoms in heteroalkyl and cycloheteroalkyl groups include, but are not limited to, —Z^{a}, halo, —O⁻, —OZ^{b}, —SZ^{b}, —S⁻, —NZ^{c}Z^{c}, trihalomethyl, —CF₃, —CN, —OCN, —SCN, —NO, —NO₂, —S(O)₂Z^{b}, —S(O₂)O⁻, —S(O₂)OZ^{b}, —OS(O₂)OZ^{b}, —OS(O₂)O⁻, —P(O)(O⁻)₂, —P(O)(OZ^{b})(O⁻), —P(O)(OZ^{b})(OZ^{b}), —C(O)Z^{b}, —C(S)Z^{b}, —C(NZ^{b})Z^{b}, —C(O)OZ^{b}, —C(S)OZ^{b}, —C(O)NZ^{c}Z^{c}, —C(NZ^{b})NZ^{c}Z^{c}, —OC(O)Z^{b}, —OC(S)Z^{b}, —OC(O)OZ^{b}, —OC(S)OZ^{b}, —NZ^{b}C(O)Z^{b}, —NZ^{b}C(S)Z^{b}, —NZ^{b}C(O)OZ^{b}, —NZ^{b}C(S)OZ^{b}, —NZ^{b}C(O)NZ^{c}Z^{c}, —NZ^{b}C(NZ^{b})Z^{b}, and —NZ^{b}C(NZ^{b})NZ^{c}Z^{c}, wherein Z^{a}, Z^{b}, and Z^{c} are as defined above.

As used herein, the term “ester” means any ester of a present compound in which any of the —COOH functions of the molecule is replaced by a —COOR function, in which the R moiety of the ester is any carbon-containing group which forms a stable ester moiety, including but not limited to alkyl, alkenyl, alkynyl, cycloalkyl, cycloalkylalkyl, aryl, arylalkyl, heterocyclyl, heterocyclylalkyl and substituted derivatives thereof. The hydrolyzable esters of the present compounds are the compounds whose carboxyls are present in the form of hydrolysable ester groups. That is, these esters are pharmaceutically acceptable and can be hydrolyzed to the corresponding carboxyl acid in vivo.

In addition to the substituents described above, alkyl, alkenyl and alkynyl groups can alternatively or in addition be substituted by C₁-C₈ acyl, C₂-C₈ heteroacyl, C₆-C₁₀ aryl, C₃-C₈ cycloalkyl, C₃-C₈ heterocyclyl, or C₅-C₁₀ heteroaryl, each of which can be optionally substituted. Also, in addition, when two groups capable of forming a ring having 5 to 8 ring members are present on the same or adjacent atoms, the two groups can optionally be taken together with the atom or atoms in the substituent groups to which they are attached to form such a ring.

“Heteroalkyl,” “heteroalkenyl,” and “heteroalkynyl” and the like are defined similarly to the corresponding hydrocarbyl (alkyl, alkenyl and alkynyl) groups, but the “hetero” terms refer to groups that contain 1-3O, S or N heteroatoms or combinations thereof within the backbone residue; thus at least one carbon atom of a corresponding alkyl, alkenyl, or alkynyl group is replaced by one of the specified heteroatoms to form, respectively, a heteroalkyl, heteroalkenyl, or heteroalkynyl group. For reasons of chemical stability, it is also understood that, unless otherwise specified, such groups do not include more than two contiguous heteroatoms except where an oxo group is present on N or S as in a nitro or sulfonyl group.

While “alkyl” as used herein includes cycloalkyl and cycloalkylalkyl groups, the term “cycloalkyl” may be used herein to describe a carbocyclic non-aromatic group that is connected via a ring carbon atom, and “cycloalkylalkyl” may be used to describe a carbocyclic non-aromatic group that is connected to the molecule through an alkyl linker.

Similarly, “heterocyclyl” may be used to describe a non-aromatic cyclic group that contains at least one heteroatom (typically selected from N, O and S) as a ring member and that is connected to the molecule via a ring atom, which may be C (carbon-linked) or N (nitrogen-linked); and “heterocyclylalkyl” may be used to describe such a group that is connected to another molecule through a linker. The heterocyclyl can be fully saturated or partially saturated, but non-aromatic. The sizes and substituents that are suitable for the cycloalkyl, cycloalkylalkyl, heterocyclyl, and heterocyclylalkyl groups are the same as those described above for alkyl groups. The heterocyclyl groups typically contain 1, 2 or 3 heteroatoms, selected from N, O and S as ring members; and the N or S can be substituted with the groups commonly found on these atoms in heterocyclic systems. As used herein, these terms also include rings that contain a double bond or two, as long as the ring that is attached is not aromatic. The substituted cycloalkyl and heterocyclyl groups also include cycloalkyl or heterocyclic rings fused to an aromatic ring or heteroaromatic ring, provided the point of attachment of the group is to the cycloalkyl or heterocyclyl ring rather than to the aromatic/heteroaromatic ring.

As used herein, “acyl” encompasses groups comprising an alkyl, alkenyl, alkynyl, aryl or arylalkyl radical attached at one of the two available valence positions of a carbonyl carbon atom, and heteroacyl refers to the corresponding groups wherein at least one carbon other than the carbonyl carbon has been replaced by a heteroatom chosen from N, O and S.

Acyl and heteroacyl groups are bonded to any group or molecule to which they are attached through the open valence of the carbonyl carbon atom. Typically, they are C₁-C₈ acyl groups, which include formyl, acetyl, pivaloyl, and benzoyl, and C₂-C₈ heteroacyl groups, which include methoxyacetyl, ethoxycarbonyl, and 4-pyridinoyl.

Similarly, “arylalkyl” and “heteroarylalkyl” refer to aromatic and heteroaromatic ring systems which are bonded to their attachment point through a linking group such as an alkylene, including substituted or unsubstituted, saturated or unsaturated, cyclic or acyclic linkers. Typically the linker is C₁-C₈ alkyl. These linkers may also include a carbonyl group, thus making them able to provide substituents as an acyl or heteroacyl moiety. An aryl or heteroaryl ring in an arylalkyl or heteroarylalkyl group may be substituted with the same substituents described above for aryl groups. Preferably, an arylalkyl group includes a phenyl ring optionally substituted with the groups defined above for aryl groups and a C₁-C₄ alkylene that is unsubstituted or is substituted with one or two C₁-C₄ alkyl groups or heteroalkyl groups, where the alkyl or heteroalkyl groups can optionally cyclize to form a ring such as cyclopropane, dioxolane, or oxacyclopentane. Similarly, a heteroarylalkyl group preferably includes a C₅-C₆ monocyclic heteroaryl group that is optionally substituted with the groups described above as substituents typical on aryl groups and a C₁-C₄ alkylene that is unsubstituted or is substituted with one or two C₁-C₄ alkyl groups or heteroalkyl groups, or it includes an optionally substituted phenyl ring or C₅-C₆ monocyclic heteroaryl and a C₁-C₄ heteroalkylene that is unsubstituted or is substituted with one or two C₁-C₄ alkyl or heteroalkyl groups, where the alkyl or heteroalkyl groups can optionally cyclize to form a ring such as cyclopropane, dioxolane, or oxacyclopentane.

Where an arylalkyl or heteroarylalkyl group is described as optionally substituted, the substituents may be on either the alkyl or heteroalkyl portion or on the aryl or heteroaryl portion of the group. The substituents optionally present on the alkyl or heteroalkyl portion are the same as those described above for alkyl groups generally; the substituents optionally present on the aryl or heteroaryl portion are the same as those described above for aryl groups generally.

“Arylalkyl” groups as used herein are hydrocarbyl groups if they are unsubstituted, and are described by the total number of carbon atoms in the ring and alkylene or similar linker. Thus a benzyl group is a C7-arylalkyl group, and phenylethyl is a C8-arylalkyl.

“Heteroarylalkyl” as described above refers to a moiety comprising an aryl group that is attached through a linking group, and differs from “arylalkyl” in that at least one ring atom of the aryl moiety or one atom in the linking group is a heteroatom selected from N, O and S. The heteroarylalkyl groups are described herein according to the total number of atoms in the ring and linker combined, and they include aryl groups linked through a heteroalkyl linker; heteroaryl groups linked through a hydrocarbyl linker such as an alkylene; and heteroaryl groups linked through a heteroalkyl linker. Thus, for example, C7-heteroarylalkyl would include pyridylmethyl, phenoxy, and N-pyrrolylmethoxy.

“Alkylene” as used herein refers to a divalent hydrocarbyl group; because it is divalent, it can link two other groups together. Typically it refers to —(CH₂)ₙ— where n is 1-8 and preferably n is 1-4, though where specified, an alkylene can also be substituted by other groups, and can be of other lengths, and the open valences need not be at opposite ends of a chain.

In general, any alkyl, alkenyl, alkynyl, acyl, or aryl or arylalkyl group that is contained in a substituent may itself optionally be substituted by additional substituents. The nature of these substituents is similar to those recited with regard to the primary substituents themselves if the substituents are not otherwise described.

“Amino” as used herein refers to —NH₂, but where an amino is described as “substituted” or “optionally substituted”, the term includes NR′R″ wherein each R′ and R″ is independently H, or is an alkyl, alkenyl, alkynyl, acyl, aryl, or arylalkyl group, and each of the alkyl, alkenyl, alkynyl, acyl, aryl, or arylalkyl groups is optionally substituted with the substituents described herein as suitable for the corresponding group; the R′ and R″ groups and the nitrogen atom to which they are attached can optionally form a 3- to 8-membered ring which may be saturated, unsaturated or aromatic and which contains 1-3 heteroatoms independently selected from N, O and S as ring members, and which is optionally substituted with the substituents described as suitable for alkyl groups or, if NR′R″ is an aromatic group, it is optionally substituted with the substituents described as typical for heteroaryl groups.

As used herein, the term “carbocycle,” “carbocyclyl,” or “carbocyclic” refers to a cyclic ring containing only carbon atoms in the ring, whereas the term “heterocycle” or “heterocyclic” refers to a ring comprising a heteroatom. The carbocyclyl can be fully saturated or partially saturated, but non-aromatic. For example, the carbocyclyl encompasses cycloalkyl. The carbocyclic and heterocyclic structures encompass compounds having monocyclic, bicyclic or multiple ring systems; and such systems may mix aromatic, heterocyclic, and carbocyclic rings. Mixed ring systems are described according to the ring that is attached to the rest of the compound being described.

As used herein, the term “heteroatom” refers to any atom that is not carbon or hydrogen, such as nitrogen, oxygen or sulfur. When it is part of the backbone or skeleton of a chain or ring, a heteroatom must be at least divalent, and will typically be selected from N, O, P, and S.

As used herein, the term “alkanoyl” refers to an alkyl group covalently linked to a carbonyl (C═O) group. The term “lower alkanoyl” refers to an alkanoyl group in which the alkyl portion of the alkanoyl group is C₁-C₆. The alkyl portion of the alkanoyl group can be optionally substituted as described above. The term “alkylcarbonyl” can alternatively be used. Similarly, the terms “alkenylcarbonyl” and “alkynylcarbonyl” refer to an alkenyl or alkynyl group, respectively, linked to a carbonyl group.

As used herein, the term “alkoxy” refers to an alkyl group covalently linked to an oxygen atom; the alkyl group can be considered as replacing the hydrogen atom of a hydroxyl group. The term “lower alkoxy” refers to an alkoxy group in which the alkyl portion of the alkoxy group is C₁-C₆. The alkyl portion of the alkoxy group can be optionally substituted as described above. As used herein, the term “haloalkoxy” refers to an alkoxy group in which the alkyl portion is substituted with one or more halo groups.

As used herein, the term “sulfo” refers to a sulfonic acid (—SO₃H) substituent. As used herein, the term “sulfamoyl” refers to a substituent with the structure —S(O₂)NH₂, wherein the nitrogen of the NH₂ portion of the group can be optionally substituted as described above. As used herein, the term “sulfonamido” refers to a moiety represented by the general formula —N(X₁)—S(O₂)—X₂, wherein X₁ and X₂ are hydrogen, lower alkyl, or substituted lower alkyl. The term “sulfonate” refers to a moiety represented by the general structure —S(O₂)—OX₁, wherein X₁ is hydrogen, lower alkyl, or substituted lower alkyl. The term “sulfinyl” refers to a moiety of the general structure —S(O)—OX₁, wherein X₁ is hydrogen, lower alkyl, or substituted lower alkyl.

As used herein, the term “carboxyl” refers to a group of the structure —C(O₂)H. As used herein, the term “carbamyl” refers to a group of the structure —C(O₂)NH₂, wherein the nitrogen of the NH₂ portion of the group can be optionally substituted as described above. As used herein, the terms “monoalkylaminoalkyl” and “dialkylaminoalkyl” refer to groups of the structure -Alk₁-NH-Alk₂ and -Alk₁-N(Alk₂)(Alk₃), wherein Alk₁, Alk₂, and Alk₃ refer to alkyl groups as described above. As used herein, the term “alkylsulfonyl” refers to a group of the structure —S(O)₂-Alk wherein Alk refers to an alkyl group as described above. The terms “alkenylsulfonyl” and “alkynylsulfonyl” refer analogously to sulfonyl groups covalently bound to alkenyl and alkynyl groups, respectively. The term “arylsulfonyl” refers to a group of the structure —S(O)₂—Ar wherein Ar refers to an aryl group as described above. The term “aryloxyalkylsulfonyl” refers to a group of the structure —S(O)₂-Alk-O—Ar, where Alk is an alkyl group as described above and Ar is an aryl group as described above. The term “arylalkylsulfonyl” refers to a group of the structure —S(O)₂-AlkAr, where Alk is an alkyl group as described above and Ar is an aryl group as described above. As used herein, the term “alkyloxycarbonyl” refers to an ester substituent including an alkyl group wherein the carbonyl carbon is the point of attachment to the molecule. An example is ethoxycarbonyl, which is CH₃CH₂OC(O)—. Similarly, the terms “alkenyloxycarbonyl,” “alkynyloxycarbonyl,” and “cycloalkylcarbonyl” refer to similar ester substituents including an alkenyl group, alkenyl group, or cycloalkyl group respectively. Similarly, the term “aryloxycarbonyl” refers to an ester substituent including an aryl group wherein the carbonyl carbon is the point of attachment to the molecule. Similarly, the term “aryloxyalkylcarbonyl” refers to an ester substituent including an alkyl group wherein the alkyl group is itself substituted by an aryloxy group.

As used herein, the term “thiocarbonyl” and combinations of substituents including “thiocarbonyl” include a carbonyl group in which a double-bonded sulfur replaces the normal double-bonded oxygen in the group. The term “alkylidene” and similar terminology refer to an alkyl group, alkenyl group, alkynyl group, or cycloalkyl group, as specified, that has two hydrogen atoms removed from a single carbon atom so that the group is double-bonded to the remainder of the structure. The term “amido” refers to amino-substituted carbonyl groups that include a —CONH moiety; the nitrogen of this moiety can be further substituted, typically with a lower alkyl group. In general, the term “amido” does not include imides, which may be unstable. The term “nitro” refers to a moiety of the structure —NO₂.

The compounds described herein may contain one or more chiral centers and/or double bonds and therefore, may exist as stereoisomers, such as double-bond isomers (i.e., geometric isomers such as E and Z), enantiomers or diastereomers. The invention includes each of the isolated stereoisomeric forms (such as the enantiomerically pure isomers, the E and Z isomers, and other alternatives for stereoisomers) as well as mixtures of stereoisomers in varying degrees of chiral purity or percentage of E and Z, including racemic mixtures, mixtures of diastereomers, and mixtures of E and Z isomers, unless a specific stereoisomer is specified. Accordingly, the chemical structures depicted herein encompass all possible enantiomers and stereoisomers of the illustrated compounds including the stereoisomerically pure form (e.g., geometrically pure, enantiomerically pure or diastereomerically pure) and enantiomeric and stereoisomeric mixtures. Enantiomeric and stereoisomeric mixtures can be resolved into their component enantiomers or stereoisomers using separation techniques or chiral synthesis techniques well known to the skilled artisan. The invention includes each of the isolated stereoisomeric forms as well as mixtures of stereoisomers in varying degrees of chiral purity, including racemic mixtures. It also encompasses the various diastereomers. Other structures may appear to depict a specific isomer, but that is merely for convenience, and is not intended to limit the invention to the depicted isomer. When the chemical name does not specify the isomeric form of the compound, it denotes any one of the possible isomeric forms or mixtures of those isomeric forms of the compound. Stereoisomers, such as enantiomers, can be designated herein by conventional designations such as D- or L- for sugars or amino acids, or R- and S- for other organic compounds according to the conventional Cahn-Ingold-Prelog priority rules for designation of enantiomers.

The compounds may also exist in several tautomeric forms, and the depiction herein of one tautomer is for convenience only, and is also understood to encompass other tautomers of the form shown. Accordingly, the chemical structures depicted herein encompass all possible tautomeric forms of the illustrated compounds. The term “tautomer” as used herein refers to isomers that change into one another with great ease so that they can exist together in equilibrium; the equilibrium may strongly favor one of the tautomers, depending on stability considerations. For example, ketone and enol are two tautomeric forms of one compound.

As used herein, the term “solvate” means a compound formed by solvation (the combination of solvent molecules with molecules or ions of the solute), or an aggregate that consists of a solute ion or molecule, i.e., a compound of the invention, with one or more solvent molecules. When water is the solvent, the corresponding solvate is “hydrate.” Examples of hydrate include, but are not limited to, hem ihydrate, monohydrate, dihydrate, trihydrate, hexahydrate, and other water-containing species. It should be understood by one of ordinary skill in the art that the pharmaceutically acceptable salt, and/or prodrug of the present compound may also exist in a solvate form. The solvate is typically formed via hydration which is either part of the preparation of the present compound or through natural absorption of moisture by the anhydrous compound of the present invention.

The compounds disclosed herein, including the compounds of Formula (I), Formula (II), Formula (III), Formula (IV), Formula (V), Formula (VI), Formula (VII), Formula (VIII), Formula (IX), Formula (X), Formula (XI), Formula (XII), Formula (XIII), or derivatives thereof as described above, may exist as salts at physiological pH ranges or other ranges. Such salts are described further below. In general, the term “pharmaceutically acceptable salts” is meant to include salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either net or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either net or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isbutyric, oxalic, maleic, malonic, benzoic, succinic, suberic, fumeric, mandelic, phthalic, benzenesulfonic, p-tolylsulfonic, citric, tartaric, methanesulfonic, and the like. Also included are salts of amino acids such as arginate and the like, and salts of organic acids like glucuronic or galacturonic acids and the like (see, for example, Berge, S. M., et al., “Pharmaceutical Salts”, Journal of Pharmaceutical Science, 1977, 66, 1-19). Certain specific compounds of the present inventions contain both basic and acidic functionalities that allow the compounds to be converted into either base or acid addition salts.

In another alternative, an epigenetic modulator according to the present invention can be administered as a prodrug. As used herein, the term “prodrug” refers to compounds that are transformed in vivo to yield a disclosed compound or a pharmaceutically acceptable form of the compound. In some embodiments, a prodrug is a compound that may be converted under physiological conditions or by solvolysis to a biologically active compound as described herein. Thus, the term “prodrug” refers to a precursor of a biologically active compound that is pharmaceutically acceptable. A prodrug can be inactive when administered to a subject, but is then converted in vivo to an active compound, for example, by hydrolysis (e.g., hydrolysis in blood or a tissue). In certain cases, a prodrug has improved physical and/or delivery properties over a parent compound from which the prodrug has been derived. The prodrug often offers advantages of solubility, tissue compatibility, or delayed release in a mammalian organism (H. Bundgard, Design of Prodrugs (Elsevier, Amsterdam, 1988), pp. 7-9, 21-24), incorporated herein by this reference. A discussion of prodrugs is provided in T. Higuchi et al., “Pro-Drugs as Novel Delivery Systems,” ACS Symposium Series, Vol. 14 and in E. B. Roche, ed., Bioreversible Carriers in Drug Design (American Pharmaceutical Association & Pergamon Press, 1987), both incorporated herein by this reference. Exemplary advantages of a prodrug can include, but are not limited to, its physical properties, such as enhanced water solubility for parenteral administration at physiological pH compared to the parent compound, enhanced absorption from the digestive tract, or enhanced drug stability for long-term storage.

The term “prodrug” is also meant to include any covalently bonded carriers which release the active compound in vivo when the prodrug is administered to a subject. Prodrugs of a therapeutically active compound, as described herein, can be prepared by modifying one or more functional groups present in the therapeutically active compound in such a way that the modifications are cleaved, either in routine manipulation or in vivo, to yield the parent therapeutically active compound. Prodrugs include compounds wherein a hydroxy, amino, or mercapto group is covalently bonded to any group that, when the prodrug of the active compound is administered to a subject, cleaves to form a free hydroxy, free amino, or free mercapto group, respectively. Examples of prodrugs include, but are not limited to, formate or benzoate derivatives of an alcohol or acetamide, formamide or benzamide derivatives of a therapeutically active agent possessing an amine functional group available for reaction, and the like.

For example, if a therapeutically active agent or a pharmaceutically acceptable form of a therapeutically active agent contains a carboxylic acid functional group, a prodrug can comprise an ester formed by the replacement of the hydrogen atom of the carboxylic acid group with a group such as C₁₋₈ alkyl, C₂₋₁₂ alkanoyloxymethyl, 1-(alkanoyloxy)ethyl having from 4 to 9 carbon atoms, 1-methyl-1-(alkanoyloxy)ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, γ-butyrolacton-4-yl, di-N,N(C₁-C₂)alkylamino(C₂-C₃)alkyl (such as (3-dimethylaminoethyl), carbamoyl-(C₁-C₂)alkyl, N,N-di (C₁-C₂)alkylcarbamoyl-(C₁-C₂)alkyl and piperidino-, pyrrolidino-, or morpholino(C₂-C₃)alkyl.

Similarly, if a disclosed compound or a pharmaceutically acceptable form of the compound contains an alcohol functional group, a prodrug can be formed by the replacement of the hydrogen atom of the alcohol group with a group such as (C₁-C₆)alkanoyloxymethyl, 1-((C₁-C₆))alkanoyloxy)ethyl, 1-methyl-1-((C₁-C₆)alkanoyloxy)ethyl (C₁-C₆)alkoxycarbonyloxymethyl, N(C₁-C₆)alkoxycarbonylaminomethyl, succinoyl, (C₁-C₆)alkanoyl, α-amino(C₁-C₄)alkanoyl, arylacyl and α-aminoacyl, or α-aminoacyl-α-aminoacyl, where each α-aminoacyl group is independently selected from the naturally occurring L-amino acids, P(O)(OH)₂, P(O)(O(C₁-C₆)alkyl)₂ or glycosyl (the radical resulting from the removal of a hydroxyl group of the hemiacetal form of a carbohydrate).

If a disclosed compound or a pharmaceutically acceptable form of the compound incorporates an amine functional group, a prodrug can be formed by the replacement of a hydrogen atom in the amine group with a group such as R-carbonyl, RO-carbonyl, NRR′-carbonyl where R and R′ are each independently (C₁-C₁₀)alkyl, (C₃-C₇)cycloalkyl, benzyl, or R-carbonyl is a natural α-aminoacyl or natural α-aminoacyl-natural α-aminoacyl, C(OH)C(O)OY¹ wherein Y¹ is H, (C₁-C₆)alkyl or benzyl, C(OY²)Y³ wherein Y² is (C₁-C₄) alkyl and Y³ is (C₁-C₆)alkyl, carboxy(C₁-C₆)alkyl, amino(C₁-C₄)alkyl or mono-N or di-N,N(C₁-C₆)alkylaminoalkyl, C(Y⁴)Y⁵ wherein Y⁴ is H or methyl and Y⁵ is mono-N or di-N,N(C₁-C₆)alkylamino, morpholino, piperidin-1-yl or pyrrolidin-1-yl.

The use of prodrug systems is described in T. Järvinen et al., “Design and Pharmaceutical Applications of Prodrugs” in Drug Discovery Handbook (S. C. Gad, ed., Wiley-Interscience, Hoboken, N.J., 2005), ch. 17, pp. 733-796, incorporated herein by this reference.

Therefore, within the scope of the invention are prodrugs of a compound selected from the group consisting of a compound of Formula (I), Formula (II), Formula (III), Formula (IV), Formula (V), Formula (VI), Formula (VII), Formula (VIII), Formula (IX), Formula (X), Formula (XI), Formula (XII), and Formula (XIII) as described above.

Typically, epigenetic modulators according to the present invention are administered orally. However, in another alternative, they can be administered by another route, such as by intravenous administration, parenteral administration, intraperitoneal administration, transcutaneous administration, subcutaneous administration, or intramuscular administration.

Suitable dosages for administration of therapeutically active epigenetic modulators according to the present invention, including but not limited to the compounds of Formula (I), Formula (II), Formula (III), Formula (IV), Formula (V), Formula (VI), Formula (VII), Formula (VIII), Formula (IX), Formula (X), Formula (XI), Formula (XII), Formula (XIII), or derivatives thereof as described above, are from 0.1 mg/kg to 100 mg/kg. As detailed further below, such quantities are typically administered daily.

Typically, therapeutically active epigenetic modulators according to the present invention are administered daily. However, administration at more or less frequent intervals, such as twice or three times daily or once every two or three days, can also be used. It will be appreciated that the actual dosages of the agents used in the compositions of this invention will vary according to the particular agent being used, the particular composition formulated, the mode of administration and the particular site, host and disease and/or condition being treated. Actual dosage levels of the active ingredients in the pharmaceutical compositions of the present invention can be varied so as to obtain an amount of the active ingredient which is effective to achieve the desired therapeutic response for a particular subject, composition, and mode of administration, without being toxic to the subject. The selected dosage level depends upon a variety of pharmacokinetic factors including the activity of the particular therapeutic agent, the route of administration, the time of administration, the rate of excretion of the particular compound being employed, the severity of the condition, other health considerations affecting the subject, and the status of liver and kidney function of the subject. It also depends on the response to administration of the agents, including factors such as blood sugar level or the level of glycated hemoglobin, body mass index as well as levels of enzymes involved in liver disease such as ALT and AST. It also depends on the duration of the treatment, other drugs, compounds and/or materials used in combination with the particular therapeutic agent employed, as well as the age, weight, condition, general health and prior medical history of the subject being treated, and like factors. Methods for determining optimal dosages are described in the art, e.g., Remington: The Science and Practice of Pharmacy, Mack Publishing Co., 20^{th} ed., 2000.

Another aspect of the present invention is the use of a therapeutically active epigenetic modulator, according to the present invention, including but not limited to the compounds of Formula (I), Formula (II), Formula (III), Formula (IV), Formula (V), Formula (VI), Formula (VII), Formula (VIII), Formula (IX), Formula (X), Formula (XI), Formula (XII), Formula (XIII), or derivatives thereof to promote weight loss or stabilization of weight. As obesity is strongly linked to the development of insulin resistance and type 2 diabetes, the use of these agents to promote weight loss or stabilization of weight can be considered as prophylactic against the development of type 2 diabetes. Accordingly, this aspect is a prophylactic method for prevention of type 2 diabetes comprising the step of administering an effective quantity of an epigenetic modulator that modulates expression of at least one gene associated with type 2 diabetes to a subject to promote weight loss or weight stabilization and/or control or reverse chronic liver disease in the subject. The epigenetic modulator is as described above. The method can further comprise administration of an effective quantity of at least one additional anti-diabetic agent; suitable additional anti-diabetic agents are as described above.

Although therapeutically active epigenetic modulators according to the present invention, including but not limited to the compounds of Formula (I), Formula (II), Formula (III), Formula (IV), Formula (V), Formula (VI), Formula (VII), Formula (VIII), Formula (IX), Formula (X), Formula (XI), Formula (XII), Formula (XIII), or derivatives thereof can be administered as pure compounds, it is generally preferred to administer them as pharmaceutical compositions.

In general, a pharmaceutical composition according to the present invention for treatment of type 2 diabetes comprises:

(1) a therapeutically effective quantity of an epigenetic modulator as described above; and

(2) at least one pharmaceutically acceptable excipient, wherein the pharmaceutically acceptable excipient typically is selected from the group consisting of:
(a) a preservative;
(b) a sweetening agent;
(c) a thickening agent;
(d) a buffer;
(e) a liquid carrier;
(f) an isotonic agent;
(g) a wetting, solubilizing, or emulsifying agent;
(h) an acidifying agent;
(i) an antioxidant;
(j) an alkalinizing agent;
(k) a carrying agent;
(l) a chelating agent;
(m) a colorant;
(n) a complexing agent;
(o) a solvent;
(p) a suspending and or viscosity-increasing agent;
(q) a flavor or perfume;
(r) an oil;
(s) a penetration enhancer;
(t) a polymer;
(u) a stiffening agent;
(v) a protein;
(w) a carbohydrate;
(x) a bulking agent; and
(y) a lubricating agent.

Typically, the pharmaceutical composition is formulated for a route of administration of the pharmaceutical composition selected from the group consisting of oral administration, intravenous administration, parenteral administration, intraperitoneal administration, transcutaneous administration, subcutaneous administration, and intramuscular administration. Preferably, the pharmaceutical composition is formulated for a route of administration of the pharmaceutical composition selected from the group consisting of oral administration and intraperitoneal administration. More preferably, the pharmaceutical composition is formulated for oral administration

Typically, the at least one pharmaceutically acceptable excipient is selected from the group consisting of: a liquid carrier; an isotonic agent; a wetting, solubilizing, or emulsifying agent; a preservative; a buffer; an acidifying agent; an antioxidant; an alkalinizing agent; a carrying agent; a chelating agent; a colorant; a complexing agent; a solvent; a suspending and/or viscosity-increasing agent; a flavor or perfume; an oil; a penetration enhancer; a polymer; a stiffening agent; a thickening agent; a sweetening agent; a protein; a carbohydrate; a bulking agent; and a lubricating agent. Pharmaceutically acceptable excipients may be added to facilitate manufacture, enhance stability, control release, enhance product characteristics, enhance bioavailability, drug absorption or solubility, optimize other pharmacokinetic considerations, optimize the pharmaceutical formulation for a route of administration, enhance patient acceptability, or for another reason related to manufacture, storage, or use of a pharmaceutical composition. Excipients used in pharmaceutical compositions according to the present invention are compatible with the pharmaceutically active agent or agents included in the pharmaceutical composition, are compatible with other excipients included in the pharmaceutical composition, and are not injurious to and are tolerated by any patients to whom the pharmaceutical composition is administered.

As is generally known in the art of pharmaceutical formulation, a particular excipient can fulfill one or more of these functions in a particular pharmaceutical composition, depending on the concentration of the excipient, the other excipients in the composition, the physical form of the composition, the concentration of active agent in the composition, the intended route of administration of the composition, and other factors. The recitation of a particular excipient in a category below is not intended to exclude the possible use of the excipient in another category or categories.

Typically, the liquid carrier can be, but is not limited to, a liquid carrier selected from the group consisting of saline, phosphate buffered saline, glycerol, and ethanol.

Typically, the isotonic agent can be, but is not limited to, a polyalcohol selected from the group consisting of mannitol and sorbitol, sodium chloride, and potassium chloride.

Typically, the wetting or emulsifying agent is a surfactant. Typically, the surfactant is selected from the group consisting of benzalkonium chloride, benzethonium chloride, cetylpyridinium chloride, docusate sodium, nonoxynol 9, nonoxynol 10, octoxynol 9, poloxamer, polyoxyl 35 castor oil, polyoxyl 40, hydrogenated castor oil, polyoxyl 50 stearate, polyoxyl 10 oleyl ether, polyoxyl 20, cetostearyl ether, polyoxyl 40 stearate, polysorbate 20, polysorbate 40, polysorbate 60, polysorbate 80, sodium lauryl sulfate, sorbitan monolaureate, sorbitan monooleate, sorbitan monopalmitate, sorbitan monostearate, tyloxapol, acacia, cholesterol, diethanolamine, glyceryl monostearate, lanolin alcohols, lecithin, mono- and di-glycerides, monoethanolamine (adjunct), oleic acid (adjunct), oleyl alcohol (stabilizer), poloxamer, polyoxyethylene 50 stearate, polyoxyl 35 castor oil, polyoxyl 40 hydrogenated castor oil, polyoxyl 10 oleyl ether, polyoxyl 20 cetostearyl ether, polyoxyl 40 stearate, polysorbate 20, polysorbate 40, polysorbate 60, polysorbate 80, propylene glycol diacetate, propylene glycol monostearate, sodium lauryl sulfate, sodium stearate, sorbitan monolaurate, sorbitan monooleate, sorbitan monopalmitate, sorbitan monostearate, stearic acid, triethanolamine, emulsifying wax, cetomacrogol, and cetyl alcohol.

Typically, the preservative is selected from the group consisting of benzalkonium chloride, benzalkonium chloride solution, benzethonium chloride, benzoic acid, benzyl alcohol, butylparaben, cetylpyridinium chloride, chlorobutanol, chlorocresol, cresol, dehydroacetic acid, diazolidinyl urea, ethylparaben, methylparaben, methylparaben sodium, phenol, phenylethyl alcohol, phenylmercuric acetate, phenylmercuric nitrate, potassium benzoate, potassium sorbate, propylparaben, propylparaben sodium, sodium benzoate, sodium dehydroacetate, sodium propionate, sorbic acid, thimerosal, and thymol.

Typically, the buffer is selected from the group consisting of acetic acid, ammonium carbonate, ammonium phosphate, boric acid, citric acid, lactic acid, phosphoric acid, potassium citrate, potassium metaphosphate, potassium phosphate monobasic, sodium acetate, sodium citrate, sodium lactate solution, dibasic sodium phosphate, monobasic sodium phosphate, sodium bicarbonate, Tris (Tris(hydroxymethyl)aminomethane), MOPS (3-(N-morpholino)propanesulfonic acid), HEPES (N-(2-hydroxyethyl)piperazine-N′-(2-ethanesulfonic acid), ACES (2-[(2-amino-2-oxoethyl)amino]ethanesulfonic acid), ADA (N-(2-acetamido)2-iminodiacetic acid), AMPSO (3-[(1,1-dimethyl-2-hydroxyethylamino]-2-propanesulfonic acid), BES (N,N-bis(2-hydroxyethyl)-2-aminoethanesulfonic acid, Bicine (N,N-bis(2-hydroxyethylglycine), Bis-Tris (bis-(2-hydroxyethyl)imino-tris(hydroxymethyl)methane, CAPS (3-(cyclohexylamino)-1-propanesulfonic acid), CAPSO (3-(cyclohexylamino)-2-hydroxy-1-propanesulfonic acid), CHES (2-(N-cyclohexylamino)ethanesulfonic acid), DIPSO (3-[N, N-bis(2-hydroxyethylamino]-2-hydroxy-propanesulfonic acid), HEPPS (N-(2-hydroxyethylpiperazine)-N′-(3-propanesulfonic acid), HEPPSO (N-(2-hydroxyethyl)piperazine-N′-(2-hydroxypropanesulfonic acid), MES (2-(N-morpholino)ethanesulfonic acid), triethanolamine, imidazole, glycine, ethanolamine, phosphate, MOPSO (3-(N-morpholino)-2-hydroxypropanesulfonic acid), PIPES (piperazine-N,N′-bis(2-ethanesulfonic acid), POPSO (piperazine-N, N′-bis(2-hydroxypropaneulfonic acid), TAPS (N-tris[hydroxymethyl)methyl-3-aminopropanesulfonic acid), TAPSO (3-[N-tris(hydroxymethyl)methylamino]-2-hydroxy-propanesulfonic acid), TES (N-tris(hydroxymethyl)methyl-2-aminoethanesulfonic acid), tricine (N-tris(hydroxymethyl)methylglycine), 2-amino-2-methyl-1,3-propanediol, and 2-amino-2-methyl-1-propanol.

Typically, the acidifying agent is selected from the group consisting of acetic acid, citric acid, fumaric acid, hydrochloric acid, diluted hydrochloric acid, malic acid, nitric acid, phosphoric acid, diluted phosphoric acid, sulfuric acid, and tartaric acid.

Typically, the antioxidant is selected from the group consisting of ascorbic acid, ascorbyl palm itate, butylated hydroxyanisole, butylated hydroxytoluene, hypophosphorous acid, monothioglycerol, propyl gallate, sodium ascorbate, sodium bisulfite, sodium formaldehyde sulfoxylate, sodium metabisulfite, sodium thiosulfate, sulfur dioxide, and tocopherol.

Typically, the alkalinizing agent is selected from the group consisting of strong ammonia solution, ammonium carbonate, diethanolamine, diisopropanolamine, potassium hydroxide, sodium bicarbonate, sodium borate, sodium carbonate, sodium hydroxide, and trolamine.

Typically, the carrying agent is selected from the group consisting of acacia syrup, aromatic syrup, aromatic elixir, cherry syrup, cocoa syrup, orange syrup, syrup, corn oil, mineral oil, peanut oil, sesame oil, bacteriostatic sodium chloride for injection and bacteriostatic water for injection.

Typically, the chelating agent is selected from the group consisting of edetate disodium, ethylenediaminetetraacetic acid, citric acid, and salicylates.

Typically, the coloring agent is selected from the group consisting of ferric oxides red, yellow, black or blends, FD&C Red No. 3, FD&C Red No. 20, FD&C Yellow No. 6, FD&C Blue No. 2, D&C Green No. 5, D&C Orange No. 5, D&C Red No. 8, and dyes suitable for pharmaceutical use.

Typically, the complexing agent is selected from the group consisting of ethylenediaminetetraacetic acid, salts of ethylenediaminetetraacetic acid, gentisic acid ethanolamide, and oxyquinoline sulfate.

Typically, the solvent is selected from the group consisting of acetone, ethanol, diluted alcohol, amylene hydrate, benzyl benzoate, butyl alcohol, carbon tetrachloride, chloroform, corn oil, cottonseed oil, ethyl acetate, glycerol, hexylene glycol, isopropyl alcohol, methyl isobutyl ketone, mineral oil, oleic acid, peanut oil, polyethylene glycol, propylene carbonate, propylene glycol, sesame oil, water for injection, sterile water for injection, sterile water for irrigation, and purified water.

Typically, the suspending and/or viscosity-increasing agent is selected from the group consisting of acacia, agar, alginic acid, aluminum monostearate, bentonite, purified bentonite, magma bentonite, carbomers, carbomer 934p, carboxymethylcellulose calcium, carboxymethylcellulose sodium, carboxymethycellulose sodium 12, carrageenan, microcrystalline and carboxymethylcellulose sodium cellulose, dextrin, gelatin, guar gum, hydroxyethyl cellulose, hydroxypropyl cellulose, hydroxypropyl methylcellulose, magnesium aluminum silicate, methylcellulose, pectin, polyethylene oxide, polyvinyl alcohol, povidone, propylene glycol alginate, silicon dioxide, colloidal silicon dioxide, sodium alginate, tragacanth, Veegum, and xanthan gum.

Typically, the flavor or perfume is selected from the group consisting of anise oil, cinnamon oil, menthol, anethole, benzaldehyde, ethyl vanillin, menthol, methyl salicylate, monosodium glutamate, orange flower oil, peppermint, peppermint oil, peppermint spirit, rose oil, stronger rose water, thymol, tolu balsam tincture, vanilla, vanilla tincture, and vanillin.

Typically, the oil is selected from the group consisting of arachis oil, mineral oil, olive oil, sesame oil, cottonseed oil, safflower oil, corn oil, and soybean oil.

Typically, the penetration enhancer is selected from the group consisting of monohydroxy or polyhydroxy alcohols, mono- or polyvalent alcohols, saturated or unsaturated fatty alcohols, saturated or unsaturated fatty esters, saturated or unsaturated dicarboxylic acids, essential oils, phosphatidyl derivatives, cephalin, terpenes, amides, ethers, ketones, and ureas.

Typically, the polymer is selected from the group consisting of cellulose acetate, alkyl celluloses, hydroxyalkylcelluloses, acrylic polymers and copolymers, polyesters, polycarbonates, and polyanhydrides.

Typically, the stiffening agent is selected from the group consisting of hydrogenated castor oil, cetostearyl alcohol, cetyl alcohol, cetyl esters wax, hard fat, paraffin, polyethylene excipient, stearyl alcohol, emulsifying wax, white wax, and yellow wax.

Typically, the sweetening agent is selected from the group consisting of aspartame, dextrates, dextrose, excipient dextrose, fructose, glycerol, mannitol, propylene glycol, saccharin, calcium saccharin, sodium saccharin, sorbitol, and solution sorbitol.

Typically, the protein is selected from the group consisting of bovine serum albumin, human serum albumin (HSA), recombinant human albumin (rHA), gelatin, and casein.

Typically, the carbohydrate is selected from the group consisting of fructose, maltose, galactose, glucose, D-mannose, sorbose, lactose, trehalose, cellobiose, raffinose, melezitose, maltodextrins, dextrans, starches, mannitol, maltitol, lactitol, xylitol, sorbitol, and myoinositol.

Typically, the bulking agent is selected from the group consisting of polypeptides and amino acids.

Typically, the lubricating agent is selected from the group consisting of magnesium stearate, stearic acid, sodium lauryl sulfate, and talc.

In one alternative, a pharmaceutical composition according to the present invention is formulated for oral administration. In another alternative, a pharmaceutical composition according to the present invention is formulated for administration by injection.

Excipients for a pharmaceutical composition according to the present invention are selected such that they do not interfere with the activity of the epigenetic modulator or derivative thereof that is included in the pharmaceutical composition. Excipients for a pharmaceutical composition according to the present invention are also selected so that they do not interfere with the activity of other excipients or cause phase separation in the composition.

For example, in general, when a hydrophobic excipient such as an oil is included in the composition, a surfactant, wetting agent, or emulsifier is also included in the composition to ensure that phase separation does not occur and to ensure that composition remains stable and homogeneous. The quantities of any excipient included in a composition according to the present invention can be determined by one of ordinary skill in the art in order to ensure suitable physical properties of the composition and also in order to ensure suitable pharmacokinetics for the epigenetic modulator or derivative thereof included in the composition.

Oral dosage forms are either solid, gel or liquid. The solid dosage forms are tablets, capsules, granules, and bulk powders. Types of oral tablets include compressed, chewable lozenges and tablets which may be enteric-coated, sugar-coated or film-coated. Capsules may be hard or soft gelatin capsules, while granules and powders may be provided in non-effervescent or effervescent form with the combination of other ingredients known to those skilled in the art.

In certain embodiments, the formulations are solid dosage forms such as for example, capsules or tablets. The tablets, pills, capsules, troches and the like can contain one or more of the following ingredients, or compounds of a similar nature: a binder; a lubricant; a diluent; a glidant; a disintegrating agent; a coloring agent; a sweetening agent; a flavoring agent; a wetting agent; an enteric coating; and a film coating. Examples of binders include microcrystalline cellulose, gum tragacanth, glucose solution, acacia mucilage, gelatin solution, molasses, polvinylpyrrolidine, povidone, crospovidones, sucrose and starch paste. Lubricants include talc, starch, magnesium or calcium stearate, lycopodium and stearic acid. Diluents include, for example, lactose, sucrose, starch, kaolin, salt, mannitol and dicalcium phosphate. Glidants include, but are not limited to, colloidal silicon dioxide. Disintegrating agents include croscarmellose sodium, sodium starch glycolate, alginic acid, corn starch, potato starch, bentonite, methylcellulose, agar and carboxymethylcellulose. Coloring agents include, for example, any of the approved certified water soluble FD and C dyes, mixtures thereof; and water insoluble FD and C dyes suspended on alumina hydrate. Sweetening agents include sucrose, lactose, mannitol and artificial sweetening agents such as saccharin, and any number of spray dried flavors. Flavoring agents include natural flavors extracted from plants such as fruits and synthetic blends of compounds which produce a pleasant sensation, such as, but not limited to peppermint and methyl salicylate. Wetting agents include propylene glycol monostearate, sorbitan monooleate, diethylene glycol monolaurate and polyoxyethylene laural ether. Enteric-coatings include fatty acids, fats, waxes, shellac, ammoniated shellac and cellulose acetate phthalates. Film coatings include hydroxyethylcellulose, sodium carboxymethylcellulose, polyethylene glycol 4000 and cellulose acetate phthalate.

The epigenetic modulator can be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

When the dosage unit form is a capsule, it can contain, in addition to material of the above type, a liquid carrier such as a fatty oil. In addition, dosage unit forms can contain various other materials which modify the physical form of the dosage unit, for example, coatings of sugar and other enteric agents. The compounds can also be administered as a component of an elixir, suspension, syrup, wafer, sprinkle, chewing gum or the like. A syrup may contain, in addition to the active compounds, sucrose as a sweetening agent and certain preservatives, dyes, colorings and flavors.

The active materials, such as the epigenetic modulator as described above, can also be mixed with other active materials which do not impair the desired action, or with materials that supplement the desired action. The active ingredient is a compound or acceptable derivative thereof as described herein. Higher concentrations, up to about 98% by weight of the active ingredient may be included.

In all embodiments of pharmaceutical compositions according to the present invention, tablets and capsules formulations may be coated as known by those of skill in the art in order to modify or sustain dissolution of the active ingredient. Thus, for example, they may be coated with a conventional enterically digestible coating, such as phenyl salicylate, waxes and cellulose acetate phthalate.

Liquid oral dosage forms include aqueous solutions, emulsions, suspensions, solutions and/or suspensions reconstituted from non-effervescent granules and effervescent preparations reconstituted from effervescent granules. Aqueous solutions include, for example, elixirs and syrups. Emulsions are either oil-in-water or water-in-oil.

Elixirs are clear, sweetened, hydroalcoholic preparations. Vehicles used in elixirs include solvents. Syrups are concentrated aqueous solutions of a sugar, for example, sucrose, and may contain a preservative. An emulsion is a two-phase system in which one liquid is dispersed in the form of small globules throughout another liquid. Vehicles used in emulsions are non-aqueous liquids, emulsifying agents and preservatives. Suspensions use suspending agents and preservatives. Substances used in non-effervescent granules, to be reconstituted into a liquid oral dosage form, include diluents, sweeteners and wetting agents. Substances used in effervescent granules, to be reconstituted into a liquid oral dosage form, include organic acids and a source of carbon dioxide. Coloring and flavoring agents are used in all of the above dosage forms.

Solvents include glycerin, sorbitol, ethyl alcohol and syrup. Examples of preservatives include glycerin, methyl and propylparaben, benzoic acid, sodium benzoate and alcohol. Examples of non-aqueous liquids utilized in emulsions include mineral oil and cottonseed oil. Examples of emulsifying agents include gelatin, acacia, tragacanth, bentonite and surfactants such as polyoxyethylene sorbitan monooleate. Suspending agents include sodium carboxymethylcellulose, pectin, tragacanth, Veegum and acacia. Sweetening agents include sucrose, syrups, glycerin and artificial sweetening agents such as saccharin. Wetting agents include propylene glycol monostearate, sorbitan monooleate, diethylene glycol monolaurate and polyoxyethylene lauryl ether. Organic acids include citric and tartaric acid. Sources of carbon dioxide include sodium bicarbonate and sodium carbonate. Coloring agents include any of the approved certified water soluble FD and C dyes, and mixtures thereof. Flavoring agents include natural flavors extracted from plants such as fruits, and synthetic blends of compounds which produce a pleasant taste sensation.

For a solid dosage form, the solution or suspension, in for example, propylene carbonate, vegetable oils or triglycerides, is in some embodiments encapsulated in a gelatin capsule. Such solutions, and the preparation and encapsulation thereof, are disclosed in U.S. Pat. Nos. 4,328,245; 4,409,239; and 4,410,545. For a liquid dosage form, the solution, e.g., for example, in a polyethylene glycol, may be diluted with a sufficient quantity of a liquid vehicle, e.g., water, to be easily measured for administration.

Alternatively, liquid or semi-solid oral formulations may be prepared by dissolving or dispersing the active compound or salt in vegetable oils, glycols, triglycerides, propylene glycol esters (e.g., propylene carbonate) and other such carriers and encapsulating these solutions or suspensions in hard or soft gelatin capsule shells. Other useful formulations include those set forth in U.S. Pat. Nos. RE28,819 and 4,358,603. Briefly, such formulations include, but are not limited to, those containing a compound provided herein, a dialkylated mono- or poly-alkylene glycol, including, but not limited to, 1,2-dimethoxyethane, diglyme, triglyme, tetraglyme, polyethylene glycol-350-dimethyl ether, polyethylene glycol-550-dimethyl ether, polyethylene glycol-750-dimethyl ether wherein 350, 550 and 750 refer to the approximate average molecular weight of the polyethylene glycol, and one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, thiodipropionic acid and its esters, and dithiocarbamates.

Other formulations include, but are not limited to, aqueous alcoholic solutions including an acetal. Alcohols used in these formulations are any water-miscible solvents having one or more hydroxyl groups, including, but not limited to, propylene glycol and ethanol. Acetals include, but are not limited to, di(lower alkyl) acetals of lower alkyl aldehydes such as acetaldehyde diethyl acetal.

In another alternative, a pharmaceutical composition according to the present invention can further comprise an effective quantity of at least one additional anti-diabetic therapeutic agent. Such additional anti-diabetic agents are described further below. Pharmaceutical compositions according to the present invention can also be prepared in a number of physical forms. The physical form of the composition can be selected by one of ordinary skill in the art for administration and depends on the quantity of epigenetic modulator or derivative thereof, the presence or absence and, if present, the quantity of other therapeutically effective components, the excipient or excipients used, and the route of administration. Suitable physical forms include, but are not limited to, solutions, suspensions, gels, quick dissolve powders, quick dissolve tablets, capsules, tablets, multiple capsules, multiple tablets, chewables, bars, and other forms.

When a pharmaceutical composition according to the present invention is in the physical form of a capsule or tablet, the composition can include as an excipient a binding material, such as but not limited to, block polymers, natural and synthetic rubber, polyacrylates, polyurethanes, silicones, polysiloxanes and styrene-butadiene copolymers. The composition can also include as an excipient a plasticizer, such as, but not limited to, diethyl phthalate and glycerol. The composition can also include as an excipient a tablet or capsule diluent, such as, but not limited to, dibasic calcium phosphate, kaolin, lactose, mannitol, microcrystalline cellulose, powdered cellulose, precipitated calcium carbonate, sodium carbonate, sodium phosphate, sorbitol, and starch. The composition can also include as an excipient a tablet or capsule opaquant, such as, but not limited to, titanium dioxide.

When a pharmaceutical composition according to the present invention is in the physical form of a tablet, the composition can include as an excipient a tablet anti-adherent agent, such as, but not limited to, magnesium stearate and talc. The composition can also include as an excipient a tablet binder, such as, but not limited to, acacia, alginic acid, carboxymethylcellulose sodium, compressible sugar, ethylcellulose, gelatin, liquid glucose, methylcellulose, non-crosslinked polyvinyl pyrrolidone, and pregelatinized starch. The composition can also include as an excipient a tablet coating agent such as, but not limited to, liquid glucose, hydroxyethyl cellulose, hydroxypropyl cellulose, hydroxypropyl methylcellulose, methylcellulose, ethylcellulose, cellulose acetate phthalate and shellac. The composition can also include as an excipient a tablet direct compression excipient such as, but not limited to, dibasic calcium phosphate. The composition can also include as an excipient a tablet disintegrant such as, but not limited to, alginic acid, carboxymethylcellulose calcium, microcrystalline cellulose, polacrilin potassium, cross-linked polyvinylpyrrolidone, sodium alginate, sodium starch glycolate, and starch. The composition can also include as an excipient a tablet glidant such as, but not limited to, colloidal silica, corn starch, and talc. The composition can also include as an excipient a tablet lubricant such as, but not limited to, calcium stearate, magnesium stearate, mineral oil, stearic acid, and zinc stearate. The composition can also include as an excipient a tablet polishing agent such as, but not limited to, carnauba wax and white wax.

In another embodiment of the present invention, the pharmaceutical composition can be in the physical form of a rapidly dissolving tablet. Rapidly dissolving tablets are disclosed in U.S. Pat. No. 9,273,040 to Layton et al., U.S. Pat. No. 9,220,747 to Nilsson et al., U.S. Pat. No. 9,192,580 to Green et al., U.S. Pat. No. 8,545,989 to Norman et al., U.S. Pat. No. 7,815,937 to Mezaache et al., U.S. Pat. No. 6,221,392 to Khankari et al., U.S. Pat. No. 6,024,981 to Khankari et al., U.S. Pat. No. 5,807,578 to Acosta-Cuello et al., U.S. Pat. No. 5,807,577 to Ouali, U.S. Pat. No. 5,807,576 to Allen. Jr. et al., U.S. Pat. No. 5,776,491 to Allen, Jr. et al., U.S. Pat. No. 5,709,886 to Bettman et al., U.S. Pat. No. 5,639,475 to Bettman et al., U.S. Pat. No. 5,635,210 to Allen, Jr. et al., U.S. Pat. No. 5,607,697 to Alkire et al., U.S. Pat. No. 5,595,761 to Allen, Jr. et al., U.S. Pat. No. 5,587,180 to Allen, Jr., et al., U.S. Pat. No. 5,503,846 to Wehling et al., U.S. Pat. No. 5,466,464 to Masaki et al., U.S. Pat. No. 5,401,513 to Wehling et al., U.S. Pat. No. 5,223,264 to Wehling et al., U.S. Pat. No. 5,219,574 to Wehling et al., and U.S. Pat. No. 5,178,878 to Wehling et al. Such formulations can include, for example, intrabuccally disintegrating solid formulations or preparations which comprise the active ingredient, a sugar comprising lactose and/or mannitol and 0.12% (w/w) to 1.2% (w/w), based on the solid components, of agar and which has a density of 400 mg/mL to 1,000 mg/mL and have a sufficient strength for handling, which in practice may mean sufficient strength to withstand removal from a blister packaging without disintegrating. In one alternative, these dosage forms are hard, compressed, rapidly dissolvable dosage forms adapted for direct oral dosing comprising an active ingredient and a matrix including a non-direct compression filter and a lubricant, where the dosage form is adapted to rapidly dissolve in the mouth of a patient and thereby liberate the active ingredient, and having a friability of about 2% or less when tested according to the U.S.P., the dosage form optionally having a hardness of at least about 15 Newtons (N), preferably from 15-50 N. Typically, such dosage forms dissolve in about 90 seconds or less (preferably 60 seconds or less and most preferably 45 seconds or less) in the patient's mouth. Such formulations can also include particles made of an active ingredient, such as an epigenetic modulator or derivative thereof, and a protective material in which the particles are incorporated. Typically, these particles are provided in an amount of between about 0.01 and about 75% by weight based on the weight of the tablet. The tablet also includes a matrix made from a non-direct compression filler, a wicking agent, and a hydrophobic lubricant. The tablet matrix comprises at least about 60% rapidly water soluble ingredients based on the total weight of the matrix material. The tablet has a hardness of between about 15 and about 50 Newtons, a friability of less than 2% when measured by U.S.P. and is adapted to dissolve spontaneously in the mouth of a patient in less than about 60 seconds and thereby liberate the particles and be capable of being stored in bulk. A very fine grained or powdered sugar known as a non-direct compression sugar may be used as a filler in the matrix of this embodiment of the present invention. This material, in part because of its chemical composition and in part because of its fine particle size, will dissolve readily in the mouth in a matter of seconds once it is wetted by saliva. Not only does this mean that it can contribute to the speed at which the dosage form will dissolve, it also means that while the patient is holding the dissolving dosage form in his or her mouth, the filler will not contribute a “gritty” or “sandy” texture thus adversely affecting the organoleptic sensation of taking the dosage form. In contrast, direct compression versions of the same sugar are usually granulated and treated to make them larger and better for compaction. While these sugars are water soluble, they may not be solubilized quickly enough. As a result, they can contribute to the gritty or sandy texture of the dosage form as it dissolves. Dissolution time in the mouth can be measured by observing the dissolution time of the tablet in water at about 37° C. The tablet is immersed in the water without forcible agitation or with minimal agitation. The dissolution time is the time from immersion to substantially complete dissolution of the rapidly water soluble ingredients of the tablet as determined by visual observation. Particularly preferred fillers, in accordance with the present invention are non-direct compression sugars and sugar alcohols which meet the specifications discussed above. Such sugars and sugar alcohols include, without limitation, dextrose, mannitol, sorbitol, lactose and sucrose. Of course, dextrose, for example, can exist as either a direct compression sugar, i.e., a sugar which has been modified to increase its compressibility, or a non-direct compression sugar. Generally, the balance of the formulation can be matrix. Thus the percentage of filler can approach 100%. However, generally, the amount of non-direct compression filler useful in accordance with the present invention ranges from about 25 to about 95%, preferably between about 50 and about 95% and more preferably from about 60 to about 95%. The amount of lubricant used can generally range from between about 1 to about 2.5% by weight, and more preferably between about 1.5 to about 2% by weight. Hydrophobic lubricants useful in accordance with the present invention include alkaline stearates, stearic acid, mineral and vegetable oils, glyceryl behenate and sodium stearyl fumarate. Hydrophilic lubricants can also be used. Protective materials useful in accordance with this embodiment of the present invention may include any of the polymers conventionally utilized in the formation of microparticles, matrix-type microparticles and microcapsules. Among these are cellulosic materials such as naturally occurring cellulose and synthetic cellulose derivatives; acrylic polymers and vinyl polymers. Other simple polymers include proteinaceous materials such as gelatin, polypeptides and natural and synthetic shellacs and waxes. Protective polymers may also include ethylcellulose, methylcellulose, carboxymethyl cellulose and acrylic resin material sold under the registered trade mark EUDRAGIT® by Rhone Pharma GmbH of Weiterstadt, Germany. In addition to the ingredients previously discussed, the matrix may also include wicking agents, non-effervescent disintegrants and effervescent disintegrants. Wicking agents are compositions which are capable of drawing water up into the dosage form. They help transport moisture into the interior of the dosage form. In that way the dosage form can dissolve from the inside, as well as from the outside. Any chemical which can function to transport moisture as discussed above can be considered a wicking agent. Wicking agents include a number of traditional non-effervescent disintegration agents. These include, for example, microcrystalline cellulose (AVICEL PH 200, AVICEL PH 101), Ac-Di-Sol (Croscarmellose Sodium) and PVP-XL (a crosslinked polyvinylpyrrolidone); starches and modified starches, polymers, and gum such as arabic and xanthan. Hydroxyalkyl celluloses such as hydroxymethylcellulose, hydroxypropylcellulose and hydroxypropylmethylcellulose, as well as compounds such as carbopol may be used as well. The conventional range of non-effervescent disintegrant agents used in conventional tablets can be as high as 20%. However, generally, the amount of disintegration agent used ranges from between about 2 and about 5%. Typically, the amount of wicking agents used may range from between 2 to about 12% and preferably from between 2 to about 5%. Other ingredients, such as non-effervescent disintegrants or an effervescent couple, can be included; preferred effervescent couples evolve gas by means of a chemical reaction which takes place upon exposure of the effervescent disintegration couple to water and/or to saliva in the mouth, and typically evolve gas by the reaction of a solid acid source and an alkali monohydrogen carbonate or other carbonate source. The acid sources can include, but are not limited to, citric acid, tartaric acid, malic acid, fumaric acid, adipic acid, and succinic acid. Carbonate sources include dry solid carbonate and carbonate or bicarbonate salts such as sodium bicarbonate, sodium carbonate, potassium bicarbonate, or potassium carbonate. In another alternative, a rapidly dissolving tablet can comprise one of the following alternatives (the proportions are for the non-therapeutically-active components): (i) 65-92% by weight of a polyol or mixture of polyols; 2-8% by weight of a cross-linked polyvinylpyrrolidone; 2-6% by weight of sodium croscarmellose; 3-12% by weight of starch; 0.05-0.5% by weight silica gel; and 0.05-0.5% by weight of colloidal silica; (ii) 75-90% by weight of a polyol or mixture of polyols; 3-7% by weight of a cross-linked polyvinyl pyrrolidone; 1-4% by weight of sodium croscarmellose; 4-10% by weight of starch; 0.05-0.3% by weight silica gel; and 0.05-0.3% by weight colloidal silica; (iii) 80-88% by weight of a polyol or mixture of polyols; 3.5-6% by weight of a cross-linked polyvinyl pyrrolidone; 2.5-3.5% by weight of sodium croscarmellose; 5-9% by weight of starch; 0.05-0.25% by weight silica gel; and 0.05-0.25% by weight of colloidal silica; and (iv) 84-85% by weight of a polyol or mixture of polyols; 4-5% by weight of a cross-linked polyvinyl pyrrolidone; 2.9-3.2% by weight of sodium croscarmellose; 7-8% by weight of starch; 0.15-0.20% by weight silica gel; and 0.15-0.20% by weight of colloidal silica. Suitable polyols for these alternatives include sorbitol, mannitol, maltitol, erythritol, xylitol, lactitol, and mixtures thereof. Suitable disintegrating agents include crospovidone, sodium starch glycolate, sodium croscarmellose, and mixtures thereof. Other excipients such as glidants can be included, as can coloring agents, lubricants, citric acid, ascorbic acid, and sweetening agents.

In some alternatives according to the present invention for rapidly dissolving tablets, the dosage form can include a superdisintegrant. Superdisintegrants include, but are not limited to, crospovidone, sodium croscarmellose, and sodium starch glycolate. A superdisintegrant is a disintegrant that has an Eq. Moisture content at 25° C. and 90% relative humidity of over 50%.

In some alternatives according to the present invention, the dosage form can include a high molecular weight polyethylene glycol or a polyethylene glycol glyceryl ester, such as those described in U.S. Pat. No. 7,815,937. The high molecular weight polyethylene glycol and the polyethylene glycol glyceryl ester can be incorporated into microspheres. Either the microspheres or the therapeutically active agent or agents can be coated or encapsulated with at least one coating, such as methacrylate/cellulose polymers, acrylate/cellulose polymers, ethylcellulose, hydroxypropylcellulose, hydroxypropylmethylcellulose, hydroxypropylmethylcellulose phthalate, cellulose acetate phthalate, Eudragit NE 300, Eudragit RS, or Eudragit L 30 D.

In some alternatives according to the present invention for rapidly dissolving tablets, the dosage form can include a pharmaceutically acceptable starch, a starch degrading enzyme, and a tablet lubricant.

In some alternatives according to the present invention for rapidly dissolving tablets, the dosage form can include a first polypeptide component and a second polypeptide component, wherein the first polypeptide component and the second polypeptide component have the same net charge in solution (i.e., either a negative charge or a positive charge). The first polypeptide component can comprise a non-hydrolyzed gelatin and the second polypeptide can comprise a hydrolyzed gelatin. The composition can further comprise a bulking agent.

In some alternatives according to the present invention for rapidly dissolving tablets, the dosage form includes a microencapsulated mixture of sodium bicarbonate and citric acid. The microencapsulation can be by ethylcellulose.

In some alternatives according to the present invention for rapidly dissolving tablets, the dosage form includes a sugar selected from the group consisting of lactose and mannitol and agar.

In some alternatives according to the present invention for rapidly dissolving tablets, the dosage form includes particulate magnesium carbonate and an oil absorbed thereon. The oil can be white mineral oil, soybean oil, or another vegetable oil; the oil can also include flavoring.

In another embodiment of the present invention, the pharmaceutical composition can be in the physical form of a rapidly dissolving powder. Rapidly dissolving powders are disclosed in U.S. Pat. No. 6,197,817 to Matier et al.

In some alternatives according to the present invention for rapidly dissolving powders, the dosage form includes lactose monohydrate, crospovidone, sodium bicarbonate, and magnesium stearate; sweetening agents and flavors may also be added.

In another embodiment of the present invention, the pharmaceutical composition can be in the physical form of a suspension for oral administration. Suspensions for oral administration are disclosed in U.S. Pat. No. 9,309,285 to Roberts et al., U.S. Pat. No. 9,290,491 to Dalziel et al., U.S. Pat. No. 9,284,279 to Ford et al., U.S. Pat. No. 9,283,183 to Mammen et al., and U.S. Pat. No. 9,273,005 to Mercurio et al.

In some alternatives according to the present invention for suspensions for oral administration, the dosage form includes suspending agents such as, for example, ethoxylated isostearyl alcohols, polyoxyethylene sorbitol and sorbitan esters, microcrystalline cellulose, aluminum metahydroxide, bentonite, agar-agar and tragacanth.

In some alternatives according to the present invention for suspensions for oral administration, the dosage form includes natural or synthetic gums, resins, methylcellulose, sodium carboxymethylcellulose, or another suspending agent.

In some alternatives according to the present invention for suspensions for oral administration, the dosage form includes fumaric acid, sodium chloride, methylparaben, propylparaben, granulated sugar, sorbitol, Veegum, flavoring, and coloring.

In some alternatives according to the present invention for suspensions for oral administration, the dosage form includes glycerol, sorbitol, sodium saccharin, xanthan gum, flavoring, citric acid, sodium citrate, methylparaben, and potassium sorbate.

In another embodiment of the present invention, the pharmaceutical composition can be in the physical form of a gel for oral administration. In general, a pharmaceutical composition that is in the form of a gel is liquid and includes one or more gel-forming agents.

In some alternatives according to the present invention for gels for oral administration, the gel-forming agent is selected from the group consisting of polyethylene glycol, polyacrylic acid, polyethylene oxide, polyvinyl alcohol, hydroxypropyl methyl cellulose, methylcellulose, hydroxyethyl cellulose, carboxymethyl cellulose, polyvinyl alcohol, polyvinylpyrrolidone, carbopol, gum Arabic, gum tragacanth, alginate, carrageenate, agar, gelatin, carbomers, and combinations thereof. Other gel-forming agents are known in the art and are described in V. G. Kadajji & G. V. Betageri, “Water Soluble Polymers for Pharmaceutical Applications,” Polymers 3: 1972-2009 (2011, and include polyacrylamide, poly-N-(2-hydroxypropyl)methacrylamide, divinyl ether-maleic anhydride copolymers, polyoxazoline, polyphosphoesters, polyphosphazenes, xanthan gum, pectin, chitosan derivatives, dextran, guar gum, hyaluronic acid, albumin, starch and derivatives of starch, and combinations thereof.

Other excipients as described above that are compatible with a physical form of a gel for oral administration can be included in the gel.

In another embodiment of the present invention, the pharmaceutical composition can be in the form of a chewable solid. The chewable solid can be a chewable tablet, as described in U.S. Pat. No. 9,320,741 to Bradner et al.; a chewable lozenge as described in U.S. Pat. No. 9,304,134 to Smith; a chewable gum as described in U.S. Pat. No. 9,278,091 to Johnson et al.; or a chewable bar as described in U.S. Pat. No. 9,302,017 to Sancilio et al. Other chewable dosage forms are known in the art.

In another alternative of a method according to the present invention, in addition to administration of an effective quantity of an epigenetic modulator according to the present invention, such as a compound of Formula (I), Formula (II), Formula (III), Formula (IV), Formula (V), Formula (VI), Formula (VII), Formula (VIII), Formula (IX), Formula (X), Formula (XI), Formula (XII), or Formula (XIII) or a derivative thereof, the method comprises administration of at least one additional anti-diabetic agent.

In one alternative, the additional anti-diabetic agent is a biguanide such as metformin. Biguanides are disclosed in U.S. Pat. No. 9,540,325 to Kim et al.; U.S. Pat. No. 9,481,642 to Baron et al.; U.S. Pat. No. 9,480,663 to Baron et al.; U.S. Pat. No. 9,464,042 to Kim et al.; U.S. Pat. No. 9,416,098 to Kim et al.; U.S. Pat. No. 9,321,742 to Kim et al.; U.S. Pat. No. 9,133,110 to Kim et al.; U.S. Pat. No. 9,060,941 to Lodin et al.; U.S. Pat. No. 8,796,338 to Baron; U.S. Pat. No. 8,668,931 to Kositprapa et al.; U.S. Pat. No. 8,648,111 to Kim et al.; U.S. Pat. No. 8,470,368 to Kositprapa et al.; U.S. Pat. No. 8,309,125 to Kositprapa et al.; U.S. Pat. No. 8,084,058 to Lodin et al.; U.S. Pat. No. 7,959,946 to Kositprapa et al.; U.S. Pat. No. 7,785,627 to Kositprapa et al.; U.S. Pat. No. 7,396,858 to Taka et al.; U.S. Pat. No. 7,285,681 to Moinet et al.; U.S. Pat. No. 6,693,094 to Pearson et al.; U.S. Pat. No. 6,287,586 to Orvig et al.; U.S. Pat. No. 4,028,402 to Fischer et al.; and U.S. Pat. No. 4,017,539 to Bosies et al.

In another alternative, the additional anti-diabetic agent is a sulfonylurea, such as, but not limited to, acetohexamide, carbutamide, chlorpropamide, glycyclamide, metahexamide, tolazamide, tolbutamide, glibenclamide, glibomuride, gliclazide, glipizide, gliquidone, glisoxepide, glyclopyramide, or glimepiride. Sulfonylureas are disclosed in U.S. Pat. No. 6,875,793 to Bhagwat et al.; U.S. Pat. No. 6,693,094 to Pearson et al.; U.S. Pat. No. 6,610,746 to Fryburg et al.; U.S. Pat. No. 6,537,578 to Bhagwat et al.; U.S. Pat. No. 6,099,862 to Chen; U.S. Pat. No. 6,056,977 to Bhagwat et al.; U.S. Pat. No. 5,972,973 to Whitcomb; U.S. Pat. No. 5,859,037 to Whitcomb; and U.S. Pat. No. 4,505,921 to Beregi et al.

In yet another alternative, the additional anti-diabetic agent is a thiazolidinedione, such as, but not limited to, pioglitazone or rosiglitazone. Thiazolidinediones are disclosed in U.S. Pat. No. 9,155,729 to Colca et al.; U.S. Pat. No. 9,126,959 to Colca et al.; U.S. Pat. No. 8,912,335 to Colca et al.; U.S. Pat. No. 8,668,931 to Kositprapa et al.; U.S. Pat. No. 8,470,368 to Kositprapa et al.; U.S. Pat. No. 8,383,656 to Chen et al.; U.S. Pat. No. 8,309,125 to Kositprapa et al.; U.S. Pat. No. 8,301,442 to Colca et al.; U.S. Pat. No. 8,084,058 to Lodin et al.; U.S. Pat. No. 8,067,450 to Colca et al.; U.S. Pat. No. 7,959,946 to Kositprapa et al.; U.S. Pat. No. 7,785,627 to Kositprapa et al.; U.S. Pat. No. 7,368,574 to Lynch et al.; U.S. Pat. No. 7,358,366 to Blackler et al.; U.S. Pat. No. 7,001,910 to Mourelle Mancini et al.; U.S. Pat. No. 6,815,457 to Blackler et al.; U.S. Pat. No. 6,787,551 to Hong et al.; U.S. Pat. No. 6,756,013 to Pfahl et al.; U.S. Pat. No. 6,288,096 to Andersson et al.; U.S. Pat. No. 6,130,216 to Antonucci et al.; U.S. Pat. No. 6,046,202 to Antonucci et al.; U.S. Pat. No. 5,990,139 to Yano et al.; U.S. Pat. No. 5,965,589 to Sohda et al.; U.S. Pat. No. 5,910,592 to Pool et al.; U.S. Pat. No. 5,811,439 to Ogawa et al.; U.S. Pat. No. 5,506,245 to Regnier et al.; U.S. Pat. No. 5,489,602 to Sohda et al.; U.S. Pat. No. 5,478,852 to Olefsky et al.; U.S. Pat. No. 5,478,850 to Hindley et al.; U.S. Pat. No. 5,457,109 to Antonucci et al.; U.S. Pat. No. 5,441,971 to Sohda et al.; U.S. Pat. No. 5,401,761 to Goldstein et al.; U.S. Pat. No. 5,330,999 to de Nantueil et al.; U.S. Pat. No. 5,330,998 to Clark et al.; U.S. Pat. No. 5,296,605 to de Nantueil et al.; U.S. Pat. No. 5,266,582 to de Nantueil et al.; U.S. Pat. No. 5,223,522 to Clark et al.; U.S. Pat. No. 5,130,379 to Clark et al.; U.S. Pat. No. 5,120,752 to Clark et al.; U.S. Pat. No. 5,061,717 to Clark et al.; U.S. Pat. No. 5,037,842 to Goldstein; U.S. Pat. No. 5,036,079 to Clark et al.; U.S. Pat. No. 4,725,610 to Meguro et al.; and U.S. Pat. No. 4,687,777 to Meguro et al.

In yet another alternative, the additional anti-diabetic agent is a DPP-4 inhibitor, such as, but not limited to, sitagliptin, vildagliptin, saxagliptin, linagliptin, gemigliptin, anagliptin, teneligliptin, alogliptin, trelagliptin, or omarigliptin. DPP-4 inhibitors are disclosed in U.S. Pat. No. 9,457,029 to Dugi et al.; U.S. Pat. No. 9,340,579 to Hayashida et al.; U.S. Pat. No. 8,633,190 to Goto et al.; U.S. Pat. No. 8,071,583 to Himmelsbach; U.S. Pat. No. 8,030,515 to Kim et al.; U.S. Pat. No. 7,652,021 to Aranyi et al.; U.S. Pat. No. 7,411,093 to Boehringer et al.; U.S. Pat. No. 7,235,538 to Kanstrup et al.; U.S. Pat. No. 7,192,952 to Kanstrup et al.; U.S. Pat. No. 6,869,947 to Kanstrup et al.; U.S. Pat. No. 6,645,995 to Kanstrup et al.; and U.S. Pat. No. 6,380,398 to Kanstrup et al.

In yet another alternative, the additional anti-diabetic agent is a gliflozin, such as, but not limited to, canagliflozin, dapagliflozin, or empagliflozin. Gliflozins are disclosed in U.S. Pat. No. 9,371,303 to Choi et al.; U.S. Pat. No. 9,198,925 to Bindra et al.; U.S. Pat. No. 9,034,921 to Choi; U.S. Pat. No. 9,006,197 to Lee et al.; U.S. Pat. No. 8,921,412 to Kim et al.; U.S. Pat. No. 8,685,934 to Strumpf et al.; U.S. Pat. No. 8,586,550 to Lee et al.; U.S. Pat. No. 8,514,380 to Lee et al.; U.S. Pat. No. 8,361,972 to Bindra et al.; U.S. Pat. No. 8,153,649 to Klein; U.S. Pat. No. 7,851,502 to Bindra et al.; U.S. Pat. No. 7,589,193 to Washburn et al.; U.S. Pat. No. 6,936,590 to Washburn et al.; U.S. Pat. No. 6,683,056 to Washburn et al.; U.S. Pat. No. 6,555,519 to Washburn; U.S. Pat. No. 6,515,117 to Ellsworth et al.; and U.S. Pat. No. 6,414,126 to Ellsworth et al.

In yet another alternative, the additional anti-diabetic agent is a glucagon-like peptide-1 receptor agonist such as, but not limited to, exatenide, liraglutide, lixisenatide, albiglutide, or dulaglutide. Glucagon-like peptide-1 receptor agonists are disclosed in U.S. Pat. No. 9,526,764 to Werner et al.; U.S. Pat. No. 9,408,893 to Niemoller et al.; and U.S. Pat. No. 8,877,805 to Konishi et al.

In yet another alternative, the additional anti-diabetic agent is an amylin analog, such as, but not limited to, pramlintide. Amylin analogs are disclosed in U.S. Pat. No. 8,598,120 to Soares et al.; U.S. Pat. No. 8,299,024 to Rabinovitch et al.; U.S. Pat. No. 8,263,550 to Beeley et al.; and U.S. Pat. No. 7,298,060 to Erickson et al.

These additional agents can also be administered together with therapeutically active epigenetic modulators according to the present invention, including but not limited to the compounds of Formula (I), Formula (II), Formula (III), Formula (IV), Formula (V), Formula (VI), Formula (VII), Formula (VIII), Formula (IX), Formula (X), Formula (XI), Formula (XII), Formula (XIII), or derivatives thereof, as described above, to treat insulin resistance in type 1 diabetes or prediabetes, to promote weight loss or weight stabilization and/or control or reverse chronic liver disease in subjects.

In another alternative, additional agents for treatment of chronic liver disease, especially non-alcoholic fatty liver disease, can be administered together with therapeutically active epigenetic modulators according to the present invention, including but not limited to the compounds of Formula (I), Formula (II), Formula (III), Formula (IV), Formula (V), Formula (VI), Formula (VII), Formula (VIII), Formula (IX), Formula (X), Formula (XI), Formula (XII), Formula (XIII), or derivatives thereof, as described above.

Additional agents for treatment of chronic liver disease, especially non-alcoholic fatty liver disease, include, but are not limited to, metformin, thiazolidinediones, statins, pentoxyfylline, elafibranor, and obeticholic acid. Statins include, but are not limited to, atorvastatin, fluvastatin, lovastatin, pitavastatin, pravastatin, rosuvastatin, cerivastatin, mevastatin, and simvastatin.

When multiple therapeutic agents are administered, each therapeutic agent can be administered separately, or two or more therapeutic agents can be administered in a single pharmaceutical composition. For example, when three therapeutic agents are to be administered, the following possibilities exist. (1) Each of the three therapeutic agents is administered individually; in this case, each agent can be administered in a separate pharmaceutical composition or as the agent alone without use of a pharmaceutical composition for the agent. Further details on the composition and preparation of pharmaceutical compositions are provided below. In this alternative, zero, one, two, or three separate pharmaceutical compositions can be used. (2) Two of the therapeutic agents are administered together in a single pharmaceutical composition, while the third therapeutic agent is administered separately, either as the agent alone or in a separate pharmaceutical composition. (3) All three therapeutic agents are administered together in a single pharmaceutical composition.

### ADVANTAGES OF THE INVENTION

The present invention provides methods and compositions employing small molecules that have the activity of directly enhancing insulin sensitivity through epigenetic regulation. These methods and compositions provide a new avenue for treating type 2 diabetes as well as insulin resistance in type 1 diabetes or prediabetes, obesity and chronic liver disease. They are well tolerated, do not produce significant side effects, and can be used together with other therapeutic agents, such as anti-diabetic agents.

Methods according to the present invention possess industrial applicability for the preparation of a medicament for the treatment of type 2 diabetes, and, in some alternatives, insulin resistance in type 1 diabetes or prediabetes as well as obesity and chronic liver disease. Compositions according to the present invention possess industrial applicability as pharmaceutical compositions, particularly for the treatment of type 2 diabetes, and, in some alternatives, insulin resistance in type 1 diabetes or prediabetes, obesity and chronic liver disease.

The method claims of the present invention provide specific method steps that are more than general applications of laws of nature and require that those practicing the method steps employ steps other than those conventionally known in the art, in addition to the specific applications of laws of nature recited or implied in the claims, and thus confine the scope of the claims to the specific applications recited therein. In some contexts, these claims are directed to new ways of using an existing drug.

The inventions illustratively described herein can suitably be practiced in the absence of any element or elements, limitation or limitations, not specifically disclosed herein. Thus, for example, the terms “comprising,” “including,” “containing,” etc. shall be read expansively and without limitation. In addition, unless specifically excluded, the term “comprising” shall encompass and support the terms “consisting essentially of” and “consisting of” with respect to the effect of the transitional phrase used on the scope of the claims. Additionally, the terms and expressions employed herein have been used as terms of description and not of limitation, and there is no intention in the use of such terms and expressions of excluding any equivalents of the future shown and described or any portion thereof, and it is recognized that various modifications are possible within the scope of the invention claimed. Thus, it should be understood that although the present invention has been specifically disclosed by preferred embodiments and optional features, modification and variation of the inventions herein disclosed can be resorted by those skilled in the art, and that such modifications and variations are considered to be within the scope of the inventions disclosed herein. The inventions have been described broadly and generically herein. Each of the narrower species and subgeneric groupings falling within the scope of the generic disclosure also form part of these inventions. This includes the generic description of each invention with a proviso or negative limitation removing any subject matter from the genus, regardless of whether or not the excised materials specifically resided therein.

In addition, where features or aspects of an invention are described in terms of the Markush group, those schooled in the art will recognize that the invention is also thereby described in terms of any individual member or subgroup of members of the Markush group. It is also to be understood that the above description is intended to be illustrative and not restrictive. Many embodiments will be apparent to those of in the art upon reviewing the above description. The scope of the invention should therefore, be determined not with reference to the above description, but should instead be determined with reference to the appended claims, along with the full scope of equivalents to which such claims are entitled. The disclosures of all articles and references, including patent publications, are incorporated herein by reference.

1. A method for treatment of type 2 diabetes comprising the step of administering an effective quantity of an epigenetic modulator that modulates expression of at least one gene associated with type 2 diabetes to a subject with type 2 diabetes.

2. The method of claim 1 wherein the epigenetic modulator is selected from the group consisting of a JMJD inhibitor, an HDAC inhibitor, a G9a inhibitor, a SETD7 inhibitor, and a DBP/p300 BRD inhibitor.

3. The method of claim 2 wherein the epigenetic modulator is a JMJD inhibitor.

4. The method of claim 3 wherein the JMJD inhibitor is selected from the group consisting of: 2,4-pyridinedicarboxylic acid; 3-((2-pyridin-2-yl)-6-(1,2,4,5-tetrahydro-3H-benzo[d]azepin-3-yl)pyrimidin-4-yl)amino)propanoic acid; 3-((2-pyridin-3-yl)-6-(1,2,4,5-tetrahydro-3H-benzo[d]azepin-3-yl)pyrimidin-4-yl)amino)propanoic acid; and ethyl 3-((2-pyridin-3-yl)-6-(1,2,4,5-tetrahydro-3H-benzo[d]azepin-3-yl)pyrimidin-4-yl)amino)propanoate.

5. (canceled)

6. The method of claim 2 wherein the epigenetic modulator is an HDAC inhibitor.

7. The method of claim 6 wherein the HDAC inhibitor is selected from the group consisting of: 4-(dimethylamino)-N-(6-hydroxyamino)6-oxohexyl)benzamide; N¹-hydroxy-N⁸-phenyloctanediamide; 4-4-chloro-2-methylphenoxy-N-hydroxybutanamide; pyridine-3-ylmethyl 4-(1-((2-aminophenyl)amino)vinyl)benzyl)carbamate; and 6-(1,3-dioxo-1H-benzo[de]isoquinolin-2(3H)-yl-N-hydroxyhexanamide.

8. (canceled)

9. The method of claim 2 wherein the epigenetic modulator is a G9a inhibitor.

10. The method of claim 9 wherein the G9a inhibitor is selected from the group consisting of: 5′-methoxy-6′-(3-pyrrolidin-1-yl)propoxy)spiro[cyclobutane-1,3′indol]-2′-amine; and 7-(2-(2-(dimethyl amino)ethoxy)ethoxy)-6-methoxy-2-(4-methyl-1,4-diazepan-1-yl)-N-(1-methylpiperidin-4-yl)quinazolin-4-amine, tri(trifluoroacetate) salt.

11. The method of claim 2 wherein the epigenetic modulator is a SETD7 inhibitor.

12. The method of claim 11 wherein the SETD7 inhibitor is (R)-8-fluoro-N-(1-oxo-1-(pyrrolidin-1-yl)-3-(4-(trifluoromethyl)phenyl)propan-2-yl)-1,2,3,4-tetrahydroisoquinoline-6-sulfonamide.

13. The method of claim 2 wherein the epigenetic modulator is a DBP/p300 BRD inhibitor.

14. The method of claim 13 wherein the CBP/p300 BRD inhibitor is (S)-4-(1-(2-(3-chloro-4-methoxyphenyl)-6-(3,5-dimethylisoxazol-4-yl)3λ²-benzo[d]imidazole-4-yl)propan-2-yl)morpholine.

15. The method of claim 2 wherein the epigenetic modulator is a compound selected from the group consisting of a compound of Formula (I), Formula (II), Formula (III), Formula (IV), Formula (V), Formula (VI), Formula (VII), Formula (VIII), Formula (IX), Formula (X), Formula (XI), Formula (XII), and Formula (XIII) with at least one substituent at a saturated carbon atom selected from the group consisting of C₆-C₁₀ aryl, heteroaryl containing 1-4 heteroatoms selected from N, O, and S, C₁-C₁₀ alkyl, C₁-C₁₀ alkoxy, cycloalkyl, F, amino (NR¹R²), nitro, —SR, —S(O)R, —S(O₂)R, —S(O₂)NR¹R², and —CONR¹R², which can in turn be optionally substituted.

16. (canceled)

17. The method of claim 2 wherein the epigenetic modulator is administered daily.

18. The method of claim 2 wherein the epigenetic modulator is administered at a dosage of from 0.1 mg/kg to 100 mg/kg.

19. (canceled)

20. The method of claim 2 wherein the epigenetic modulator is administered by a route selected from the group consisting of oral administration, intravenous administration, parenteral administration, intraperitoneal administration, transcutaneous administration, subcutaneous administration, and intramuscular administration.

21.-22. (canceled)

23. The method of claim 2 wherein the administration of the epigenetic modulator reduces insulin resistance.

24. The method of claim 1 wherein the method further comprises administration of an effective quantity of at least one additional anti-diabetic agent.

25. The method of claim 24 wherein the at least one additional anti-diabetic agent is selected from the group consisting of a biguanide, a sulfonylurea, a thiazolidinedione, a DPP-4 inhibitor, a gliflozin, a glucagon-like peptide-1 receptor agonist, and an amylin analog.

26.-32. (canceled)

33. The method of claim 1 wherein the epigenetic modulator is administered in a pharmaceutical composition comprising: (i) an effective quantity of the epigenetic modulator; and (ii) at least one pharmaceutically acceptable excipient.

34. The method of claim 33 wherein the at least one pharmaceutically acceptable excipient is selected from the group consisting of:
(a) a preservative;
(b) a sweetening agent;
(c) a thickening agent;
(d) a buffer;
(e) a liquid carrier;
(f) an isotonic agent;
(g) a wetting, solubilizing, or emulsifying agent;
(h) an acidifying agent;
(i) an antioxidant;
(j) an alkalinizing agent;
(k) a carrying agent;
(l) a chelating agent;
(m) a colorant;
(n) a complexing agent;
(o) a solvent;
(p) a suspending and or viscosity-increasing agent;
(q) a flavor or perfume;
(r) an oil;
(s) a penetration enhancer;
(t) a polymer;
(u) a stiffening agent;
(v) a protein;
(w) a carbohydrate;
(x) a bulking agent; and
(y) a lubricating agent.

35. The method of claim 24 wherein the epigenetic modulator and the at least one additional anti-diabetic agent are administered in a pharmaceutical composition comprising: (i) an effective quantity of the epigenetic modulator; (ii) an effective quantity of the at least one additional anti-diabetic agent; and (iii) at least one pharmaceutically acceptable excipient.

36. The method of claim 35 wherein the at least one pharmaceutically acceptable excipient is selected from the group consisting of:
(a) a preservative;
(b) a sweetening agent;
(c) a thickening agent;
(d) a buffer;
(e) a liquid carrier;
(f) an isotonic agent;
(g) a wetting, solubilizing, or emulsifying agent;
(h) an acidifying agent;
(i) an antioxidant;
(j) an alkalinizing agent;
(k) a carrying agent;
(l) a chelating agent;
(m) a colorant;
(n) a complexing agent;
(o) a solvent;
(p) a suspending and or viscosity-increasing agent;
(q) a flavor or perfume;
(r) an oil;
(s) a penetration enhancer;
(t) a polymer;
(u) a stiffening agent;
(v) a protein;
(w) a carbohydrate;
(x) a bulking agent; and
(y) a lubricating agent.

37. A pharmaceutical composition for treatment of type 2 diabetes comprising:
(a) an effective quantity of an epigenetic modulator; and
(b) at least one pharmaceutically acceptable excipient.

38. The pharmaceutical composition of claim 37 wherein the epigenetic modulator is selected from the group consisting of a JMJD inhibitor, an HDAC inhibitor, a G9a inhibitor, a SETD7 inhibitor, and a CBP/p300 BRD inhibitor.

39.-50. (canceled)

51. The pharmaceutical composition of claim 37 wherein the pharmaceutical composition is formulated for a route of administration of the pharmaceutical composition selected from the group consisting of oral administration, intravenous administration, parenteral administration, intraperitoneal administration, transcutaneous administration, subcutaneous administration, and intramuscular administration.

52.-53. (canceled)

54. The pharmaceutical composition of claim 37 wherein the pharmaceutically acceptable excipient is selected from the group consisting of:
(a) a preservative;
(b) a sweetening agent;
(c) a thickening agent;
(d) a buffer;
(e) a liquid carrier;
(f) an isotonic agent;
(g) a wetting, solubilizing, or emulsifying agent;
(h) an acidifying agent;
(i) an antioxidant;
(j) an alkalinizing agent;
(k) a carrying agent;
(l) a chelating agent;
(m) a colorant;
(n) a complexing agent;
(o) a solvent;
(p) a suspending and or viscosity-increasing agent;
(q) a flavor or perfume;
(r) an oil;
(s) a penetration enhancer;
(t) a polymer;
(u) a stiffening agent;
(v) a protein;
(w) a carbohydrate;
(x) a bulking agent; and
(y) a lubricating agent.

55.-78. (canceled)

79. The composition of claim 37 wherein the composition further comprises an effective quantity of an additional anti-diabetic agent.

80. The composition of claim 79 wherein the at least one additional anti-diabetic agent is selected from the group consisting of a biguanide, a sulfonylurea, a thiazolidinedione, a DPP-4 inhibitor, a gliflozin, a glucagon-like peptide-1 receptor agonist, and an amylin analog.

81. A prophylactic method for prevention of type 2 diabetes comprising the step of administering an effective quantity of an epigenetic modulator that modulates expression of at least one gene associated with type 2 diabetes to a subject to promote normalization of blood glucose levels in type 1 diabetes or prediabetes, promote weight loss or weight stabilization and/or reverse chronic liver disease in the subject.

82. The prophylactic method of claim 81 wherein the epigenetic modulator is selected from the group consisting of a JMJD inhibitor, an HDAC inhibitor, a G9a inhibitor, a SETD7 inhibitor, and a CBP/p300 BRD inhibitor.

83.-96. (canceled)

97. The prophylactic method of claim 82 wherein the method further comprises administration of an effective quantity of at least one additional anti-diabetic agent.

98. The prophylactic method of claim 97 wherein the at least one additional anti-diabetic agent is selected from the group consisting of a biguanide, a sulfonylurea, a thiazolidinedione, a DPP-4 inhibitor, a gliflozin, a glucagon-like peptide-1 receptor agonist, and an amylin analog.

99.-129. (canceled)
